(19) United States
(12) Patent Application Publication
(10) Pub. No.: US 2024/0136013 A1
(43) Pub. Date: Apr. 25, 2024

(54) QUANTIFICATION OF RNA MUTATION EXPRESSION

(71) Applicant: Genentech, Inc., South San Francisco, CA (US)

(72) Inventor: Andrew J. WALLACE, Hayward, CA (US)

(21) Appl. No.: 18/541,590

(22) Filed: Dec. 15, 2023

Related U.S. Application Data
(63) Continuation of application No. PCT/US22/33869, filed on Jun. 16, 2022.
(60) Provisional application No. 63/212,044, filed on Jun. 17, 2021.

(51) Int. Cl. G16B 20/20

(52) U.S. Cl. CPC G16B 20/20

(57) ABSTRACT

A method for quantifying ribonucleic acid (RNA) mutation expression. For each read pair of a read pair group, a set of contiguously aligned regions and a splice junction configuration are identified. Each read pair is within a selected range of a location of interest. Each read pair of the read pair group is classified based on the set of contiguously aligned regions and the splice junction configuration that correspond to each read pair, a reference genome, and a selected mutation. A mutation-centric output is generated for the read pair group.

### CROSS-REFERENCE TO RELATED APPLICATION

This application claims the benefits of U.S. provisional application No. 63/212,044, filed Jun. 17, 2021, the contents of which are incorporated herein by reference in its entirety for all purposes.

### BACKGROUND

Neoantigens are tumor-specific antigens derived from somatic mutations in tumors. Peptide fragments of the tumor-specific antigens are presented by a subject's cancer cells and antigen-presenting cells. Neoantigen therapies, such as, but not limited to, neoantigen vaccines, are a relatively new approach for providing individualized cancer treatment. Neoantigen vaccines can prime a subject's T cells to recognize and attack cancer cells expressing one or more particular tumor neoantigens. This approach generates a tumor-specific immune response that spares healthy cells while targeting tumor cells. The individualized vaccine may be engineered or selected based on a subject-specific tumor profile. The tumor profile can be defined by determining DNA and/or RNA sequences from a subject's tumor cell and using the sequences to identify neoantigens that are present in tumor cells but absent in normal cells.

### SUMMARY

The embodiments described herein provide methods and systems for quantifying RNA expression levels for a mutation (e.g., an indel). In one or more embodiments, the mutation is a somatic mutation that can give rise to a distinct neoantigen. The embodiments described herein provide methods and systems for classifying read pairs as being consistent with or not consistent with having the mutation. Further, the embodiments described herein provide methods and systems for quantifying read pairs that are consistent with having an isoform-specific mutation (e.g., indels). This type of quantification may be used in, for example, without limitation, the development of therapies (e.g., cancer therapeutics).

In one or more embodiments, a method is provided for quantifying ribonucleic acid (RNA) mutation expression. For each read pair of a read pair group, a set of contiguously aligned regions and a splice junction configuration are identified. Each read pair is within a selected range of a location of interest. Each read pair of the read pair group is classified based on the set of contiguously aligned regions and the splice junction configuration that correspond to each read pair, a reference genome, and a selected mutation. A mutation-centric output is generated for the read pair group.

In one or more embodiments, a method is provided for quantifying isoforms. For each read pair of a read pair group, a set of contiguously aligned regions and a splice junction configuration are identified. Each read pair is within a selected range of a location of interest. The method includes evaluating whether each read pair of the read pair group is consistent with or inconsistent with a first isoform that is derived from a transcript that includes the location of interest based on the set of contiguously aligned regions and the splice junction configuration identified for each read pair. An isoform-specific output is generated that identifies a first count for read pairs in the read pair group that is consistent with the first isoform.

In one or more embodiments, a method is provided for quantifying isoform-specific RNA mutation expression. A set of contiguously aligned regions and a splice junction configuration are identified for each read pair in a read pair group within selected range of a location of interest at which a selected mutation is expected. Each read pair in the read pair group is classified as supporting either a reference allele, an alternate allele, or a null allele based on the set of contiguously aligned regions for each read pair. Each read pair in the read pair group is classified as being either consistent with or inconsistent with an isoform in a set of isoforms that is derived from a transcript that includes the location of interest based on the set of contiguously aligned regions and the splice junction configuration for each read pair. An output is generated that includes counts that are at least one of isoform-specific or mutation-centric.

In some embodiments, a system is provided that includes one or more data processors and a non-transitory computer storage medium containing instructions which, when executed on the one or more data processors, cause the one or more data processors to perform part of all of one or more methods disclosed herein.

In some embodiments, a computer program product is provided that is tangibly embodied in a non-transitory machine-readable storage medium and that includes instructions configured to cause one or more data processors to perform part of all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part of all of one or more methods and/or part or all of one or more processes disclosed herein. Some embodiments of the present disclosure include computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 is a schematic diagram illustrating different isoforms in accordance with one or more embodiments.

FIG. 2 is a schematic diagram illustrating an example of a quantification system for quantifying RNA mutation expression in accordance with one or more embodiments.

FIG. 3 is a flow diagram illustrating an example of a process for quantifying RNA mutation expression in accordance with one or more embodiments.

FIG. 4 is a flow diagram illustrating an example of a process for classifying a read pair based on the type of allele at a location of interest in accordance with one or more embodiments.

FIG. 5 is a flow diagram of a process for quantifying RNA mutation expression in accordance with one or more embodiments.

FIG. 6 is a schematic diagram of read pairs and the transcript, first isoform, and second isoform from FIG. 1 in accordance with one or more embodiments.

FIG. 7 is a flow diagram of a process for quantifying RNA mutation expression in accordance with one or more embodiments.

FIG. 8 is an example of at least a portion of a mutation-centric output in accordance with one or more embodiments.

FIG. 9 is an example of at least a portion of an isoform-specific output in accordance with one or more embodiments.

FIG. 10 is a schematic diagram illustrating a read pair group in association with two isoforms in accordance with one or more embodiments.

FIG. 11 is a block diagram illustrating an example of a computer system in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The embodiments described herein recognize that accurate quantification of RNA expression levels may be important for various reasons. Further, the embodiments recognize that quantification of RNA expression levels at the isoform level may also be important. Still further, it may be important to quantify isoform-specific RNA expression levels with respect to a mutation. For example, quantifying isoform-specific RNA expression levels for a mutation that can give rise to a neoantigen may be important to the development of neoantigen therapies (e.g., neoantigen cancer therapies). Measuring neoantigenicity within a tumor genome may help identify which antigens will likely solicit an immune response.

Thus, the embodiments described herein provide various methods, systems, non-transitory computer readable media for quantifying RNA expression levels for a mutation (e.g., a neoantigen mutation) at a location of interest. For example, sequence information about read pairs generated for a sample (e.g., a tumor sample) may be processed. The embodiments described herein provide methods, systems, and non-transitory computer readable media for classifying the read pairs as being consistent with a reference allele (e.g., no mutation supported), consistent with an alternate allele (e.g., mutation supported), or consistent with neither the reference allele nor alternate allele.

RNA quantification using the methods, systems, and non-transitory computer readable media described herein may enable the counting of read pairs that are consistent with (or support) mutations in the form of insertions and deletions. Insertions and deletions are examples of mutations that might otherwise be eliminated from counting using some currently available methods and systems. For example, some currently available methods and systems may miss insertions and deletions in their counting, which may lead to misquantification of RNA mutation frequency (or variant allele frequency (VAF)). Further, some currently available methods and systems may miss reference alleles in their counting.

Additionally, the embodiments described herein provide methods, systems, and non-transitory computer readable media for associating the read pairs with specific isoforms. For example, a read pair may be associated with a selected isoform from a set of isoforms that is associated with the mutation. This type of quantification may be used in, for example without limitation, the development of therapeutics (e.g., cancer therapeutics). For example, this type of quantification may enable deprioritizing neoantigens derived from mutated isoforms with little to no RNA expression to provide cost and/or time savings in the development of a therapeutic. Further, RNA quantification may enable filtration of non-expressed neoantigen mutations, investigation of the determinants of expression, or both.

### II. Quantification of RNA Mutation Expression

The embodiments described herein may be generally presented with respect to the quantification of RNA expression levels for mutations that are putative neoantigen mutations (or neoantigenic). It should be understood, however, that the embodiments described herein may be similarly used to quantify RNA expression levels for other types of mutations (or variants) that give rise to other types of proteins. Further, the embodiments described herein are generally presented with respect to the processing of sequence information for read pairs, also referred to as paired-end reads. It should be understood, however, that these embodiments may be similarly used to process sequence information for individual reads.

II.A. Isoforms of an Example RNA Transcript

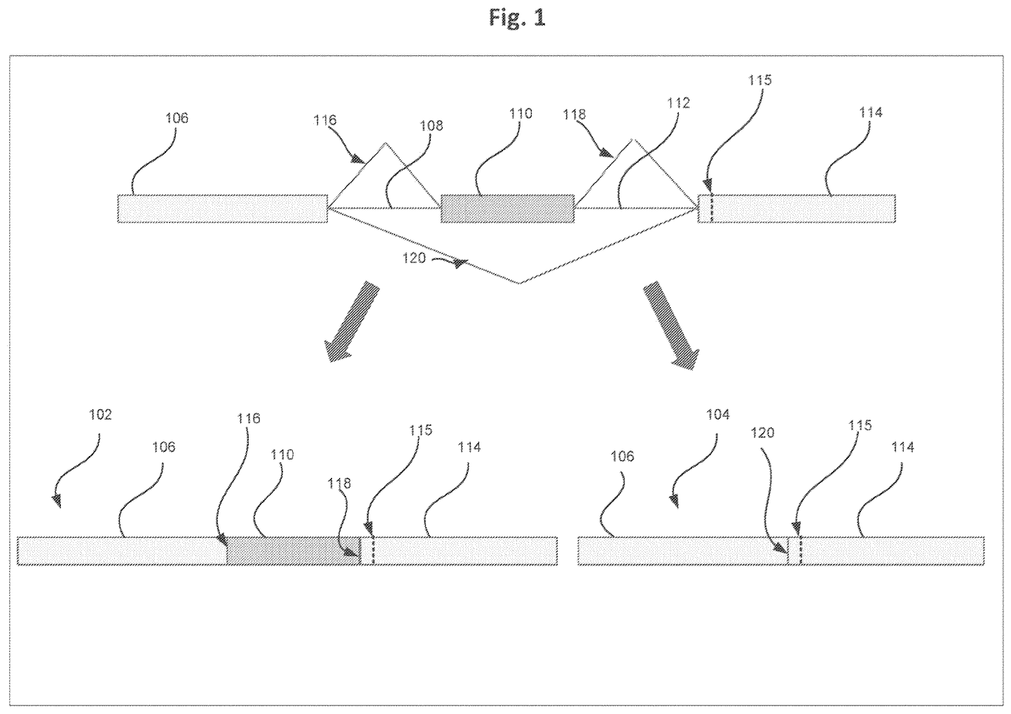

FIG. 1 is a schematic diagram illustrating different isoforms in accordance with one or more embodiments. Transcript 100 is one example of an RNA product that is formed by transcription of a DNA sequence. Transcript 100 may be referred to as a primary transcript, a precursor mRNA (pre-mRNA), or an RNA transcript. Transcript 100 may be further processed via splicing to produce mRNA (or mature mRNA). This splicing may be performed in various ways. A single transcript may be spliced in multiple ways that produce different mature mRNAs, which may be referred to as isoforms.

For example, transcript 100 may be spliced in at least two different ways to form either a first isoform 102 or a second isoform 104. Transcript 100 includes exon 106, intron 108, exon 110, intron 112, and exon 114. Location 115 is a location of interest at which a selected mutation is possible. The selected mutation can be a previously identified mutation of interest. The selected mutation may be, for example, a neoantigen mutation. For example, location 115 may be a genomic location of interest at which a neoantigen mutation has been observed previously in a population or in one or more tumor tissue samples obtained from one or more subjects. As discussed above, a neoantigen is tumor-specific antigen that is derived from one or more mutations (e.g., somatic mutations) in tumors, and is presented by a subject's cancer cells and antigen presenting cells. These types of mutations are referred to herein as neoantigen mutations.

Location 115 may span one or more nucleotides. The various possible nucleotide configurations at location 115 are referred to as alleles. A reference allele at location 115 means that the one or more nucleotides at location 115 match a reference genome that lacks the selected mutation. The reference genome may be, for example, the genome of a subject determined using healthy tissue from the subject or a genome determined from a group of healthy subjects or healthy population. Thus, the reference allele may be, for example, an allele in an unmutated state as observed in healthy tissue of the subject or in a healthy population. An alternate allele at location 115 means that the selected mutation (e.g., a putatively neoantigen mutation) is present at location 115. A null allele at location 115 means that the nucleotide configuration at location 115 matches neither the reference genome nor the selected mutation.

One form of splicing yields first isoform 102 that includes exon 106, exon 110, and exon 114. First isoform 102 has isoform splice junction 116 and isoform splice junction 118 that correspond to the removal of intron 108 and intron 112 and the joining of exon 106 with exon 110 and of exon 110 with exon 114 during the splicing of transcript 100. Another form of splicing yields second isoform 104 that includes exon 106 and exon 114, but does not include exon 110. Second isoform 104 has isoform splice junction 120 that corresponds to the removal of intron 108, exon 110, and intron 112 and the joining of exon 106 and exon 114 during the splicing of transcript 100. Isoform splice junction 116, isoform splice junction 118, and isoform splice junction 120 are also shown in relation to transcript 100.

When transcript 100 has the selected mutation at location 115, both first isoform 102 and second isoform 104 also has the selected mutation. But translation of first isoform 102 may form a different peptide (e.g., a neoantigen) than translation of second isoform 104. Because the peptides (e.g., neoantigens) produced by these different isoforms are distinct, it may be important to quantify the particular isoforms found in a biological sample. For example, it may be important to quantify RNA expression of the different isoforms found in a sample of tumor tissue to determine which one or more peptides (e.g., neoantigens) produced via translation of the different isoforms to include or exclude in the development of patient-specific immunotherapies or cancer therapies.

Quantifying RNA expression for the selected mutation (e.g., a neoantigen mutation) at location 115 includes analyzing read paus derived from at least one biological sample. The biological sample may be, for example, a diseased sample (e.g., diseased tissue, tumor tissue, etc.). The number of read pairs analyzed from the collection of read pairs generated for the biological sample may be reduced to those read pairs that are within as selected range of location 115. This type of filtering may enable reducing the overall amount of computing resources used to perform RNA expression quantification. Quantifying RNA expression for the selected mutation at location 115 may include evaluating which, if any, one or more isoforms to associate with read pairs; classifying read pairs as supporting the reference allele, the alternate allele, or null allele.

Associating a read pair with an isoform may include determining that the read pair is consistent with that isoform. For example, a read pair may be considered “consistent” with an isoform if any and all splice junctions in the read pair match corresponding isoform splice junctions in the isoform, if any and all contiguously aligned regions in the read pair are overlapped by corresponding exons in the isoform, or both. Thus, determining whether to associate a read pair with, for example, first isoform 102, second isoform 104, or both, includes performing a splice junction evaluation, an exon region evaluation, or both for the read pair.

The splice junction evaluation includes comparing a splice junction configuration generated for the read pair to the isoform splice junctions. For example, if the splice junction configuration includes two splice junctions that match to isoform splice junction 116 and isoform splice junction 118 of first isoform 102, the splice junction configuration is considered consistent with these isoform splice junctions of first isoform 102. Accordingly, the read pair is considered consistent with first isoform 102, with respect to splice junctions. If the splice junction configuration includes a single splice junction that matches to isoform splice junction 120, the splice junction configuration is considered consistent with this isoform splice junction 120 of second isoform 104. Accordingly, the read pair is considered consistent with second isoform 104, with respect to splice junctions.

The exon region evaluation includes determining whether the one or more contiguously aligned regions identified in the read pair are overlapped by the exons of the isoforms. A contiguously aligned region overlaps an exon if the genomic coordinates for the start and end of the contiguously aligned region fall within or otherwise align with the genomic coordinates for the start and end of the exon. Thus, a contiguously aligned region may overlap an exon by fully overlapping the exon such that no portion of the contiguously aligned region overlaps with an intron.

For example, if a read pair includes a first contiguously aligned region overlapped by exon 106 and a second contiguously aligned region overlapped by exon 114, the read pair is considered consistent with both first isoform 102 a second isoform 104, with respect to exon regions. If the read pair includes a first contiguously aligned region overlapped by exon 106, a second contiguously aligned region overlapped by exon 110, and a third contiguously aligned region overlapped by exon 114, the read pair can be considered consistent with first isoform 102, with respect to exon regions.

Classifying a read pair as supporting the reference allele, the alternate allele, or a null allele may depend on whether the selected mutation is an indel (e.g., insertion or deletion) or a single nucleotide substitution. If the selected mutatis is a substitution, classification includes confirming that the expected location (e.g., location 115) of the selected mutation is within a contiguously aligned region in the read pair. If the expected location is not within a contiguously aligned region of the read par, the read pair is classified as supporting the null allele if the expected location falls within a deletion or as a “skip” if not within a deletion.

If the selected mutation is an indel, the read pair is classified as supporting the reference allele when there is no alignment gap between two contiguously aligned regions of the read pair at the expected location of the selected mutation. An alignment gap is a set of nucleotides that is flanked on both sides by the two contiguously aligned regions and that does not align with the reference genome. The read pair is classified as supporting the alternate allele if an alignment gap is present between two contiguously aligned regions of the read pair at the expected location of the selected mutation and the set of nucleotides that form the alignment gap match the selected mutation. The read pair is classified as supporting a null allele if the alignment gap is present but the set of nucleotides that forms the alignment gap does not match selected mutation.

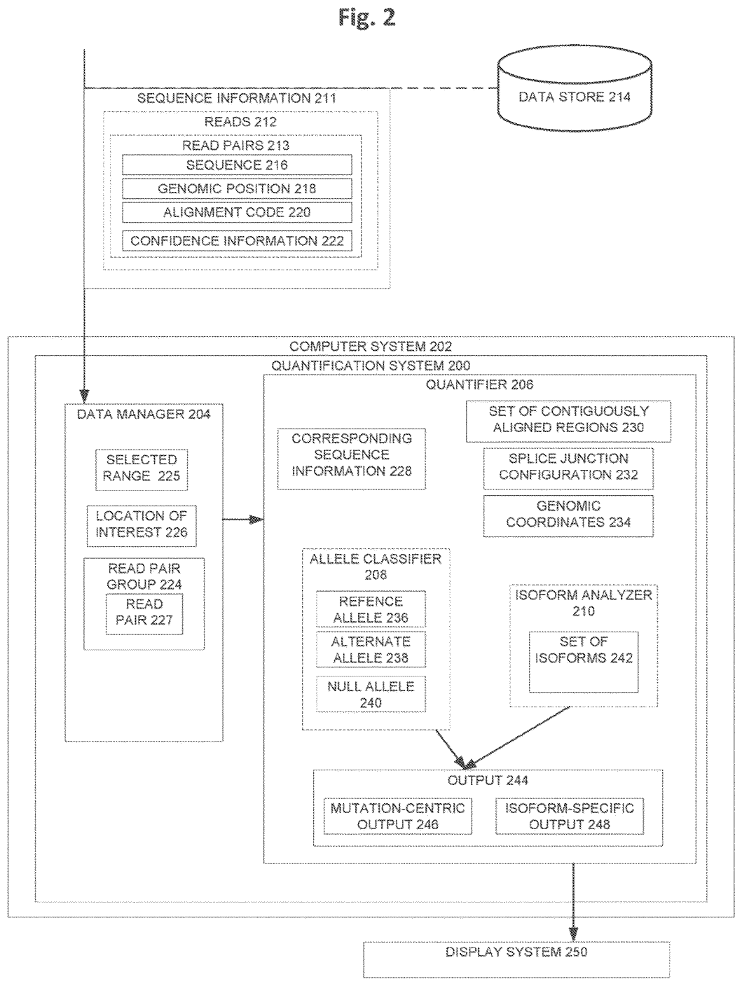

Quantification system 200 described below with respect to FIG. 2 is one example of a system that can perform RNA expression quantification. Quantification system 200 can receive read paus for a biological sample and associate these read pairs with first isoform 102 or second isoform 104, as applicable. Further, quantification system 200 can classify each read pair as supporting the reference allele, the alternate allele, or a null allele.

II.B. System for Quantifying RNA Mutation Expression

FIG. 2 is a schematic diagram illustrating an example of a quantification system 200 for quantifying RNA mutation expression in accordance with one or more embodiments. Quantification system 200 is implemented using hardware, software, firmware, or a combination thereof. Quantification system 200 may be implemented using, for example, computer system 202. Computer system 202 clades single computer or multiple computers in communication with each other. When computer system 202 includes multiple computers, in some embodiments, one computer may be located remotely with respect to at least one other computer.

Quantification system 200 includes data manager 204 and quantifier 206. Data manager 204 and quantifier 206 may be implemented using hardware, software, firmware, or combination thereof. For example, each of data 204 and quantifier 206 may be implemented as a distinct compiled computer program, interpreted language script, another type of software, or a combination thereof. In other embodiments, data manager 204 and quantifier 206 are integrated together and implemented as a single computer program, interpreted language script, other type of software, or combination thereof.

In one or more embodiments, quantifier 206 includes allele classifier 208 and isoform analyzer 210. Allele classifier 208 and isoform analyzer 210 may be separate programs. In other embodiments, allele classifier 208 and isoform analyzer 210 or the functions performed by allele classifier 208 and isoform analyzer 210 are integrated within quantifier 206. For example, the functions that would otherwise be performed by allele classifier 208 and isoform analyzer 210 may be integrated into a single program within or that is part of the program that forms quantifier 206. Further, any functions described herein as being performed by quantifier 206 may be performed by allele classifier 208, isoform analyzer 210, or both.

Quantification system 200 obtains sequence information 211 for a plurality of reads 212. Reads 212 may be obtained for a corresponding biological sample. The biological sample can be obtained from, for example, a subject (e.g., a live subject). A biological sample may be, for example, a sample of unhealthy or diseased tissue, a sample of tumor tissue, a sample of tissue that includes tumor cells, a sample of tissue that includes cancer cells, a sample of healthy or normal tissue, a sample of tissue that includes normal cells, a sample of tissue taken at a first stage or point in time during a cancer progression, a sample of tissue taken at a second stage or point in time during the cancer progression, or another type of sample.

Read 212 may be generated using, for example, one or more next-generation sequencing (NGS) systems such as, for example, without limitation, whole-exome sequencing (WES), whole genome sequencing (WGS), or both. In one or more embodiments, reads 212 may be based on RNA sequence reads. In some cases, these RNA sequence reads are mRNA sequence reads generated in at transcriptome-wide manner.

Reads 212 may be generated using, for example, paired-end sequencing such that reads 212 are paired-end reads. For example, paired-end sequencing of fragment results in two sequences, a sequence generated beginning at the 5′ end of the fragment, and a sequence generated beginning at the 3′ end of the fragment. These two sequences form a paired-end read, which may be referred to as a read pair. Accordingly, reads 212 can form read pairs 213 and sequence information 211 may be organized with respect to read pairs 213.

Quantification system 200 may obtain sequence information 211 by receiving, retrieving, or generating sequence information 211 for read pairs 213. In some embodiments, quantification system 200 retrieves sequence information 211 from data store 214. Data store 214 may include, for example, but is not limited to, at least one of a database, a data storage unit, a spreadsheet, a file, a server, a cloud storage unit, a cloud database, or some other type of data store. In some examples, data store 214 comprises one or more data storage devices separate from but in communication with compute system 202. In other examples, data store 214 is at least partially integrated as part of computers system 202.

Sequence information 211 includes various pieces of information about read pairs 213 and may be formatted in any of a number of different ways. For example, in some cases, sequence information 211 may take the form of one or more files, one or more spreadsheets, or some other type data format. In one or more embodiments, sequence information 211 includes genomic alignment information for read pairs 213. For example, sequence information 211 may include, for each read pair (e.g., paired-end read) of read pairs 213, at least one of a sequence 216, a genomic position 218, an alignment code 220, confidence information 222, some other type of information, or a combination thereof for the read pair.

Sequence 216 is the nucleotide sequence that forms the read pair. For example, sequence 216 may represent the RNA (e.g., mRNA) transcript sequence for a read pair. In one or more embodiments, the RNA's sequence may be referred to in the form of complementary DNA (cDNA) such that the RNA transcript sequence is expressed as DNA nucleotides rather than RNA nucleotides. For example, sequence 216 may represent the read pair using DNA nucleobases: A for adenine, C for cytosine, G for guanine, and T for thymine. Alternatively, sequence 216 may represent the read pair using RNA nucleobases A for adenine, C for cytosine, G for guanine, and U for uracil.

Genomic position 218 is the position (e.g., estimated position) of a read pair with respect to a genome of the subject for which reads 212 were generated. In some embodiments, this position may be denoted via a nucleotide (or corresponding base pair) position. In other embodiments, this position may be denoted by a range of nucleotides (or corresponding base pairs). As one example, the read pair may be matched to a corresponding portion of the genome to identify genomic position 218 of the read with respect to the genome.

Alignment code 220 is a code that provides alignment information about the read pair. For example, alignment code 220 may be a string of characters that provides information about the nucleotide regions that match and do not match the corresponding portion of a reference genome. In one or more embodiments, alignment code 220 is implemented as a Compact Idiosyneratic Gapped Alignment Report (CIGAR) string. CIGAR strings are explained in further detail in Section V below.

Confidence information 222 may include, for example, without limitation, a confidence score for each nucleotide in sequence 216. This confidence score for a particular nucleotide in sequence 216 indicates the confidence associated with the identification of that particular nucleotide at that position in sequence 216.

Data manager 204 processes sequence information 211 to identify read pair group 224 from read pairs 213. Read pair group 224 includes read pairs that are located within selected range 225 of, in terms of a count of sequential nucleotide positions away from, an expected location of a mutation (or variant) within the genome, which may be referred to as a location of interest 226. Selected range 225 may be, for example, without limitation, 5000 nucleotide positions long, 100,000 nucleotide positions long, or some other range between about 250 and 1,000,000 nucleotide positions long. In one or more embodiments, data manager 204 obtains selected range 225, location of interest 226, or both from data store 214.

Read pair 227 is one example of a read pair in read pair group 224. Read pair 227 is a paired-end read that has been determined to span a selected range of nucleotides or portion of the genome that includes location of interest 226. Location 115 in FIG. 1 is one example of an implementation for location of interest 226. For example, if location of interest 226 for the selected mutation is the 200,000^{th} nucleotide position of the genome, read pair 227 may be selected for inclusion within read pair group 224 if read pair 227 overlaps a portion of the genome that falls within the 175,000^{th} to 225,000^{th} nucleotide positions. Read pair 227 includes a mutation-overlapping read (a read that overlaps the 200,000^{th} nucleotide position) and its paired-end partner read or mate.

The selected mutation at location of interest 226 may take different forms including, for example, an insertion, a deletion, a substitution, etc. Accordingly, location of interest 226 may include one or more nucleotide positions. In one or more embodiments, the selected mutation is a putatively neoantigen mutation. An miRNA sequence, also referred to as a “variant-coding sequence,” that contains neoantigen mutation is a sequence that includes a sequence for a neoantigen.

Quantifier 206 receives the read pair group 224 for processing. Quantifier 206 processes corresponding sequence information 228 for read pair group 224. Corresponding sequence information 228 is the portion of sequence information 211 that corresponds to read pair group 224. In some embodiments, quantifier 206 receives corresponding sequence information 228 from data manager 204. In other embodiments, quantifier 206 itself identifies corresponding sequence information 228 for read pair group 224 from sequence information 211.

In one or more embodiments, quantifier 206 processes alignment code 220 in corresponding sequence information 228 for each read pair of read pair group 224. For example, quantifier 206 may identify a set of contiguously aligned regions, a splice junction configuration, and corresponding genomic coordinates for the set of contiguously aligned regions and the splice junction configuration for each read pair of read pair group 224.

For example, quantifier 206 may process alignment code 220 for read pair 227 to identify set of contiguously aligned regions 230 and generate splice junction configuration 232 for read pair 227. Set of contiguously aligned regions 230 includes one or more portions of read pair 227 that substantially match (e.g., exactly or nearly exactly) the genome at genomic position 218 without any alignment gaps, (e.g., insertions, deletions, etc. that do not match).

The splice junction configuration 232 for a read pair 227 identifies e presence of zero, one, or more splice junctions identified within the read pair 227 and/or the positions of any such splice junctions. A splice junction is the site of a former intron in a mature mRNA. In other words, a splice junction is a site at which an intron was removed.

In one or more embodiments, quantifier 206 parses alignment code 220, which may be, for example, a CIGAR string, into genomic coordinates 234 that can be used to identify set of contiguously aligned regions 230 and splice junction configuration 232. Genomic coordinates 234 may, for example, identify the start and end positions, with respect to the genome, of each contiguously aligned region in set of contiguously aligned regions 230 and each alignment gap (e.g., insertions, deletions) within read pair 227, as well as any splice junctions identified in splice junction configuration 232 for read pair 227.

Allele classifier 208 of quantifier 206 classifies each read pair within read pair group 224 based on the type of allele present at location of interest 226. For example, allele classifier 208 may classify each read pair of read pair group 224 as supporting reference allele 236, supporting alternate allele 238, or supporting null allele 240 (e.g., matching neither the reference allele nor the alternate allele) based on set of contiguously aligned regions 230 for each read pair. For example, read pair 227 may be classified as supporting reference allele 236 if location of interest 226 within read pair 227 matches the reference genome without mutation. Read pair 227 may be classified as supporting alternate allele 238 if location of interest 226 within read pair 227 matches the expected mutation. Read pair 227 may be classified as supporting null allele 240 at location 241 if the set of nucleotides location of interest 226 within read pair 227 matches neither the reference genome nor the expected mutation.

Allele classifier 208 counts the number of read pairs in read pair group 224 that support reference allele 236, the member of read pairs in read pair group 224 that support alternate allele 238, and the member of read pairs in read pair group 224 that support null allele 240. Allele classifier 208 performs classifying read pair group 224 and generating these counts in a manner that counts indels (e.g., insertions or deletions) as well as nucleotide substitutions.

Isoform analyzer 210 of quantifier 206 determines whether each read pair of read pair group 224 is consistent with one or more isoforms of set of isoforms 242 based on the splice junction configuration for each reach pair. Isoform analyzer 210 associates each read pair with the one or more isoforms with which that read pair has been determined to be consistent. In one or more embodiments, set of isoforms 242 includes one or more isoforms that have been identified as having the potential to give rise to a neoantigen. For example, set of isoforms 242 includes the one or more isoforms that include location of interest 226.

Each isoform in set of isoforms 242 has a set of isoform splice junctions that correspond to that isoform. The set of isoform splice junctions uniquely identifies each isoform. However, in some cases, one or more isoform splice junctions may be common to two or more isoforms in set of isoforms 242. Isoform analyzer 210 may analyze splice junction configuration 232 and genomic coordinates 234 for read pair 227 to determine whether splice junction configuration 232 can be associated with the set of isoform splice junctions associated with any of the isoforms in set of isoforms 242. Splice junction configurations 232 is consistent with a set of isoform splice junctions for an isoform if each splice junction in splice junction configuration 232 matches a corresponding isoform splice junction in the isoform.

If splice junction configuration 232 for read pair 227 is consistent with a set of isoform splice junctions for a selected isoform of set of isoforms 242, isoform analyzer 210 associates read pair 227 with that selected isoform. In other words, isoform analyzer 210 determines that read pair 227 is consistent with that selected isoform. First isoform 102 and second isoform 104 in FIG. 1 are one example of an implementation for set of isoforms 242.

Read pair 227 may be consistent with multiple isoforms of set of isoforms 242. For example, read pair 227 may include a set of splice junctions that could be consistent with multiple isoforms of set of isoforms 242. In other cases, however, read pair 227 may be exclusively consistent with a particular isoform of set of isoforms 242. For example, read pair 227 may include a set of splice junctions indicating that read pair 227 is exclusively consistent with a particular isoform.

In this manner, isoform analyzer 210 may count the number of read pairs in read pair group 224 that are consistent with at least one isoform of set of isoforms 242. Further, isoform analyzer 210 may count the number of read pairs in read pair group 224 that are exclusively consistent with a selected isoform of set of isoforms 242.

Quantifier 206 generates output 244 using the information generated by allele classifier 208, isoform analyzer 210, or both. Output 244 may include mutation-centric output 246, isoform-specific output 248, or both. Mutation-centric output 246 may include, for example, a count of the number of read pairs that support the alternate allele. Further, in some embodiments, mutation-centric output 246 may also include a count for the read pairs that support the reference allele, a count for the read pairs that support the null allele, or both. Isoform-specific output 248 may include a count for the read pairs that are consistent with each isoform of set of isoforms 242. In some embodiments, isoform-specific output 248 may include a count for the read pairs that are consistent with both a particular isoform of set of isoforms 242 and support the reference allele, a count for the read pairs that are consistent with the particular isoform and support the alternate reference allele, or both.

In various embodiments, quantification system 200 may display output 244, or at least a portion of output 244, display system 250. Output 244 may be displayed in a format (e.g., a table, a spreadsheet, a diagram, etc.) that is easily understandable to a user. In one or more embodiments, quantification system 200 is capable of processing and analyzing sequence information 211 for a plurality of selected mutations (e.g., a library or collection of known neoantigen mutations) and may generate output 244 that provides mutation-centric and isoform-specific information for each of the plurality of mutations. In some cases, this output 244 may be displayed on display system 250 in a manner that enables the information for the plurality of mutations to be viewed simultaneously.

II.C. Mutation-Centric Read Pair Classification

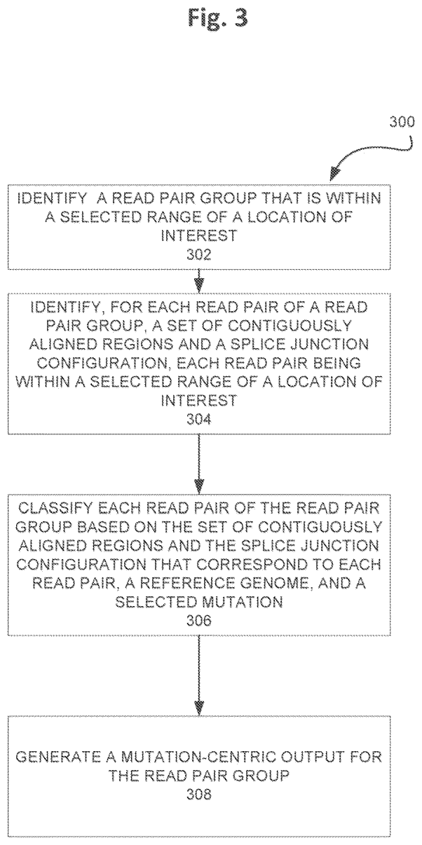

FIG. 3 is a flow diagram illustrating an example of a process for quantifying RNA mutation express in accordance with one or more embodiments. Process 300 is one example of a process that may be implemented using quantification system 200 or at least a portion of quantification system 200 in FIG. 2. For example, process 300 may be implemented using quantifier 206 in FIG. 2. In some embodiments, process 300 may be implemented using allele classifier 208, isoform analyzer 210, or both in FIG. 2.

Step 302 includes identifying a read pair group within a selected range of a location of interest. The location of interest may be, for example, a location at which a selected mutation is expected (e.g., location of interest 226 in FIG. 2, location 115 in FIG. 1, etc.). The selected mutation may be, for example, a putatively neoantigen mutation. The selected mutation may be an insertion, a deletion, a substitution, or some other type of mutation. Read pair group 224 in FIG. 2 may be one example of an implementation for the read pair group identified in step 302.

In one or more embodiments, step 302 may be performed by selecting the read pair group based on the selected and sequence information for a collection of read pairs generated via sequencing. Selected range 225 in FIG. 2 may be one example of this selected range. Sequence information 211 for read pairs 213 in in FIG. 2 may be one example of this sequence information.

Step 304 includes identifying, for each read pair of a read pair group, a set of contiguously aligned regions and a splice junction configuration, each read pair being within a selected range of a location of interest. Set of contiguously aligned regions 230 in FIG. 2 and splice junction configuration 232 in FIG. 2 are examples of implementations for the set of contiguously aligned regions and the splice junction configuration, respectively, identified for each read pair. In one or more embodiments, step 304 is performed for a given read pair by parsing the alignment code (e.g., alignment code 220 in FIG. 2) included in the portion of sequence information (e.g., sequence information 211 in FIG. 2) corresponding to the read pair. In various embodiments, step 304 further includes identifying genomic coordinates (e.g., genomic coordinates 234 in FIG. 2) that correspond with the set of contiguously aligned regions and the splice junction configuration.

Step 306 includes classifying each read pair of the read pair group based on the set of contiguously aligned regions and the splice junction configuration that correspond to each read pair, a reference genome, and a selected mutation. In one or more embodiments, step 306 includes classifying at least one read pair in the read pair group as supporting a reference allele. The reference allele matches a reference genome at the location of interest. In various embodiments, step 306 includes classifying at least one read pair in the read pair group as supporting an alternate allele. The alternate allele matches a selected mutation (e.g., a neoantigen mutation) at the location of interest. In various embodiments, step 306 includes classifying at least one read pair in the read pair group as supporting a null allele. The null allele matches neither a reference genome nor a selected mutation at the location of interest.

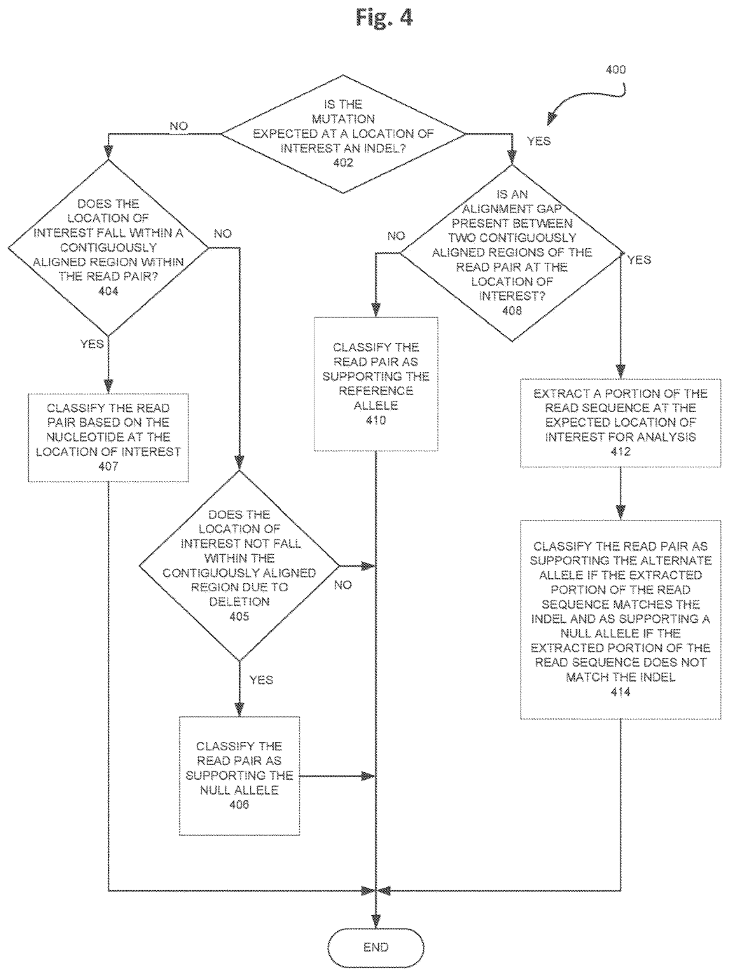

In step 306, classifying a read pair may be considered the same as classifying the allele at the location of interest within the read pair. For example, classifying the read pair as supporting the reference allele, the alternate allele, or the null allele may include classifying the allele at the location of interest as the reference allele, the alternate allele, or the null allele, respectively. Accordingly, step 306 may include identifying a first set of read pairs consistent with the reference allele, a second set of read pairs supports the alternate allele, a third set of read pairs supporting the null allele, or a combination thereof. It should be appreciated, however, that one or more other types of classifications are also possible. One example of a manner in which step 306 may be performed is described below in FIG. 4.

Step 308 includes generating a mutation-centric output for the read pair group. For example, the mutation-centric output may include a count for the number of read pairs found to support the reference allele, a count for the number of read pairs found to support the alternate allele, a count for the number of read pairs found to support the null allele, or a combination thereof.

In one or more embodiments, step 308 includes generating the mutation-centric output for the entire read pair group. In some embodiments, the mutation-centric output includes other information in addition to or in place of the counts described above. For example, the mutation-centric output may include, but is not limited to, a count for the number of read pairs in the first set of read pairs that is also consistent with least one isoform of a set of isoforms (e.g., set of isoforms 242 in FIG. 2), a count for the number of read pairs in the first set of read pairs that is consistent with no isoforms of the set of isoforms, a count for the number of read pairs in the second set of read pairs that is also consistent with at least one isoform of the set of isoforms, a count for the number of read pairs in the second set of read pairs that is consistent with no isoforms of the set of isoforms, or a combination thereof. Still further, the mutation-centric output may include a count for the number of read pairs in the read pair group that were determined to support neither the reference allele nor the alternate allele.

The mutation-centric output generated in step 308 may be used in various ways. For example, a determination may be made to include an antigen (e.g., neoantigen) that is derived from the selected mutation as a target for an immunotherapy responsive to the mutation-centric output indicating at least a threshold level of RNA expression for the selected mutation. The threshold level of RNA expression may include, for example, a threshold count of read pairs that support the alternate allele. Thus threshold count may be, for example, 5, 8, 10, 15, 20, 25, 50, 100, 200, 300, 500, 1000, 2000, or some other number of read pairs. Alternatively, a determination may be made to exclude the antigen as a target for an immunotherapy responsive to the mutation-centric output indicating that RNA expression for the selected mutation is below the threshold level. The immunotherapy may include, for example, without limitation, at least one of T cell therapy, a personalized cancer therapy, a cancer immunotherapy, an antigen-specific immunotherapy, an antigen dependent immunotherapy, a vaccine, a natural killer (NK) cell therapy, or some other type of customized therapy.

FIG. 4 is a flow diagram illustrating an example of a process for classifying a read pair based on the type of allele as a location of interest in accordance with one or more embodiments. Process 400 is one example of a process that may be implemented using quantification system 200 or at least a portion of quantification system 200 in FIG. 2. For example, process 400 may be implemented using quantifier 206 in FIG. 2. In some embodiments, process 400 may be implemented using allele classifier 208 in FIG. 2. In various embodiments, process 400 may be used to implement step 306 in FIG. 3.

Step 402 includes determining whether the mutation expected at the location of interest is an indel. As previously noted, an indel may be an insertion or a deletion. If the mutation is not an indel, the mutation is a substitution (e.g., a single nucleotide substitution) and process 400 proceeds to step 404 described below.

Step 404 includes determining whether the location of interest falls within a contiguously aligned region within the read pair. If the location of interest does not fall within a contiguously aligned region, step 405 is performed, which includes determining whether the cause of the location of interest not falling within the contiguously aligned region is due to deletion. If the location of interest does not fall within the contiguously aligned region due to deletion, step 406 is performed. Step 406 includes classifying the read pair as supporting the null allele. Otherwise, if deletion is not the reason that the location of interest does not fall within the contiguously aligned region, the location of interest likely falls within an intron, and the read pair may be classified as a “skip” with a count for the read pair being skipped and the process 400 terminating. The count is skipped because matching to either the reference allele, the alternate allele, or the null allele is not possible.

With reference again to step 404, if the location of interest does fall within a contiguously aligned region, step 407 is performed. Step 407 includes classifying the read pair based on the nucleotide at the location of interest. For example, step 407 may include matching the nucleotide at the location of interest to the reference genome of the subject from which the sample from which the read pair was derived was obtained, to the mutation, or to neither. The read pair may be classified as supporting the reference allele if the nucleotide at the location matches the corresponding nucleotide at the location of interest in the reference genome. The read pair may be classified as supporting the alternate allele if the nucleotide at the location of interest is matched to the mutation. The read pair may be classified as supporting a null allele if the nucleotide at the location of interest matches neither the nucleotide of the reference genome nor the mutation.

With reference again to step 402, if the mutation is an indel, process 400 proceeds to step 408. Step 408 includes determining whether there is an alignment gap (e.g., a non-splice junction gap) present between two contiguously aligned regions of the read pair at the location of interest. An alignment gap is a set of nucleotides that is flanked on both sides by the two contiguously aligned regions and that does not align with the reference genome. A non-splice junction gap is an alignment gap due to insertion or deletion. Step 408 is performed using the splice junction configuration for the read pair. An absence of an alignment gap may indicate that there is no insertion and no deletion at the location of interest. Accordingly, if a determination is made that there is no alignment gap present between two contiguously aligned regions of the read pair at the location of interest, step 410 is performed, which includes classifying the read pair as being consistent with the reference allele.

With reference again to step 408, if an alignment gap is present, step 412 is performed, which includes extracting a portion of the read sequence at the expected location of interest for analysis. Step 412 may be performed using, for example, string slicing. Step 414 includes classifying the read pair as supporting the alternate allele if the extracted portion of the read sequence matches the indel and as supporting a null allele if the extracted portion of the read sequence does not match the indel.

II.D. Isoform-Specific Pair Classification

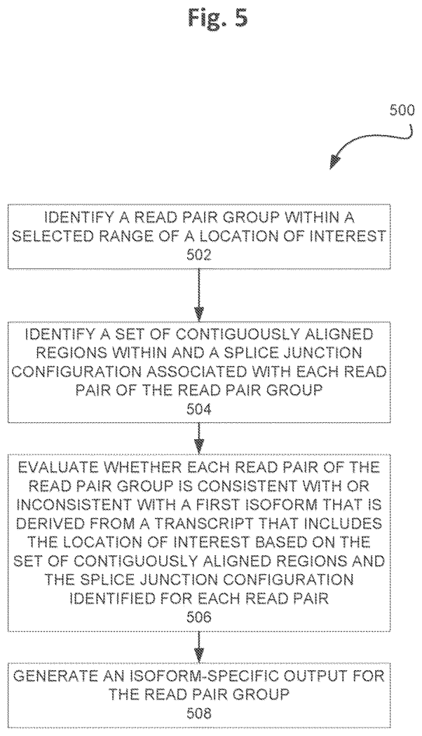

FIG. 5 is a flow diagram of a process for quantifying RNA mutation expression in accordance with one or more embodiments. Process 500 is one example of a process that may be implemented using quantification system 200 or at least a portion of quantification system 200 in FIG. 2. For example, process 500 may be implemented using quantifier 206 in FIG. 2. In some embodiments, process 400 may be implemented using allele classifier 208, isoform analyzer 210, or both in FIG. 2.

Step 502 includes identifying a read pair group within a selected range of a location of interest. The location of interest may be, for example, a location at which a selected mutation is expected (e.g., location of interest 226 in FIG. 2, location 115 in FIG. 1, etc.). The selected mutation may be, for example, a putatively neoantigen mutation. The selected mutation may be an insertion, a deletion, a substitution, or some other type of mutation. Read pair group 224 in FIG. 2 may be one example of an implementation for the read pair group identified in step 502.

In one or more embodiments, step 502 may be performed by selecting the read pair group based on the selected range and sequence information for a collection of read pairs (e.g. sequence information 211 for read pairs 213 in FIG. 2) generated via sequencing. Selected range 225 in FIG. 2 may be one example of this selected range. Sequence information 211 in FIG. 2 may be one example of this sequence information.

Step 504 includes identifying, for each read pair of a read pair group, a set of contiguously aligned regions and a splice junction configuration, each read par being within a selected range of a location of interest. Set of contiguously aligned regions 230 in FIG. 2 and splice junction configuration 232 in FIG. 2 are examples of implementations for the set of contiguously aligned regions and the splice junction configuration, respectively, identified for a given read pair. In one or more embodiments, step 504 is performed for a given read pair by parsing the alignment code (e.g., alignment code 220 in FIG. 2) included in the portion of sequence information (e.g., sequence information 211 in FIG. 2) corresponding to the read pair. In various embodiments, step 504 further includes identifying genomic coordinates (e.g., genomic coordinates 234 in FIG. 2) that correspond with the set of contiguously aligned regions and the splice junction configuration.

Step 506 includes evaluating whether each read pair of the read pair group is consistent with or inconsistent with a first isoform that is derived from a transcript that includes the location of interest based on the set of contiguously aligned regions and the splice junction configuration identified for each read pair. In one or more embodiments, step 506 includes determining that a read pair is consistent with the first isoform in response to a first determination that the splice junction configuration of the read pair of the read pair group is consistent with a set of isoform splice junctions within the isoform, in response to a second determination that the set of contiguously aligned regions within the read pair overlap (e.g., fully overlap) a set of exons within the isoform, or both.

A splice junction configuration is consistent with the set of isoform splice junctions of an isoform when all splice junctions identified by the splice junction configuration can be matched to the set of isoform splice junctions of that isoform. In some cases, a read pair has a splice junction configuration indicating that there are zero splice junctions in the read pair. Such a splice junction configuration may still be considered consistent with the set of isoform splice junctions because the splice junction configuration is not inconsistent with the set of isoform splice junctions. Said differently, the splice junction configuration of such a read pair is consistent with that isoform because the splice junction configuration does not include any splice junctions that the isoform does not also include.

Further, step 506 includes analyzing each read pair in the read pair group to determine whether a given read pair can be associated with one or more of isoforms in a set of isoforms that is derived from the transcript. The association of a read pair with an isoform is an indication that the read pair is consistent with at least that isoform. Step 506 may include, for example, associating a read pair in the read pair group with more than one of the isoforms in the set of isoforms in response to determination that the splice junction configuration is consistent with multiple isoforms. In some cases, step 506 includes associating a given read pair with a particular isoform exclusively. For example, the read pair may have a splice junction configuration that is unique to that particular isoform.

Step 508 includes generating an isoform-specific output that identifies a member of read pairs within the read pair group that are associated with the isoform. In one or more embodiments, step 508 includes generating an isoform-specific output that identifies counts for the read pair group with respect to a set of isoforms derived from the transcript. For example, the isoform-specific output may include a count of the number of read pairs consistent with the isoform, a count of the number of read pairs consistent with the isoform and the reference allele, the number of read pairs consistent with the isoform and the alternate allele, or a combination thereof.

The isoform-specific output generated in step 508 may be used in various ways. For example, a determination may be made to include an antigen (e.g., neoantigen) that is derived from a particular isoform as a target for an immunotherapy responsive to the isoform-specific output indicating at least a threshold level of RNA expression for the particular isoform. The threshold level of RNA expression may include, for example, a threshold count of read pairs that are consistent with the particular isoform. This threshold count may be, for example, 5, 8, 10, 15, 20, 25, 50, 100, 200, 300, 500, 1000, 2000, or some other number of read pairs. Alternatively, a determination may be made to exclude the antigen as a target for an immunotherapy responsive to the isoform-specific output indicating that RNA expression for the particular isoform is below the threshold level. The immunotherapy may include, for example, without limitation, at least one of a T cell therapy, a personalized cancer therapy, a cancer immunotherapy, an antigen-specific immunotherapy, an antigen-dependent immunotherapy, a vaccine, a natural killer (NK) cell therapy, or some other of customized therapy.

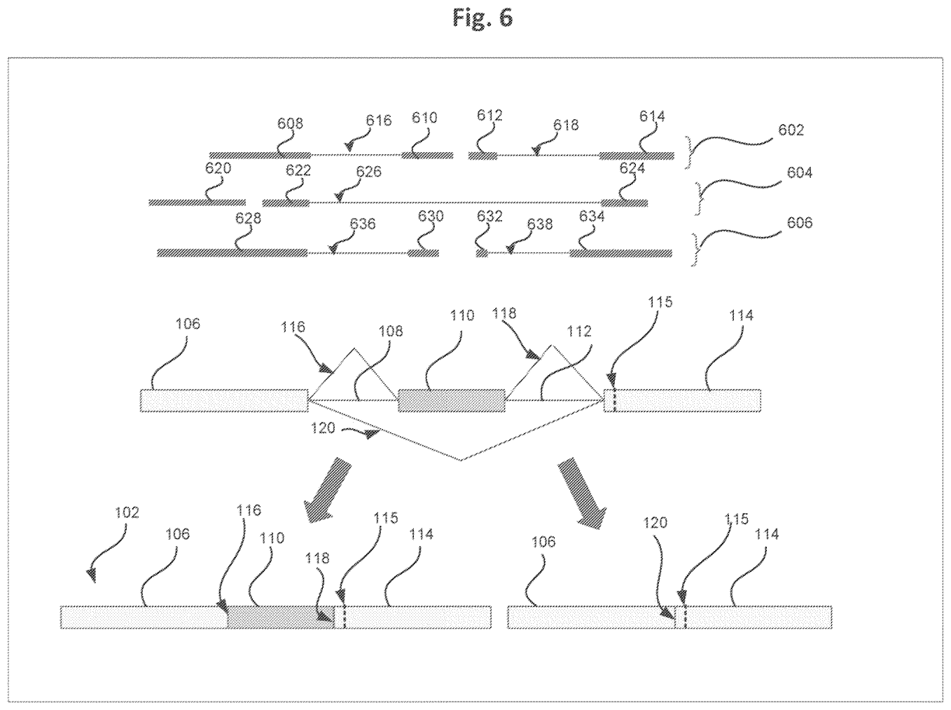

FIG. 6 is a schematic diagram of read pairs and transcript 100, first isoform 102, and second isoform 104 from FIG. 1 in accordance with one or more embodiments. Quantification system 200 in FIG. 2 may be used to accurately associate a first read pair 602, second read pair 604, and third read pair 606 with the corresponding one of first isoform 102 or second isoform 104. This association may be performed using, for example, process 500 in FIG. 5.

First read pair 602 includes contiguously aligned region 608, contiguously aligned region 610, contiguously aligned region 612, and contiguously aligned region 614 and splice junction 616 and splice junction 618. Second read pair 604 include contiguously aligned region 620, contiguously aligned region 622, and contiguously aligned 124 and splice junction 626. Third read pair 606 includes contiguously aligned region 628, contiguously aligned region 630, contiguously aligned region 632, and contiguously aligned region 634 and splice junction 636 and splice junction 638. First read pair 602, second read pair 604, and third read pair 606 are examples of implementations for a portion of read pairs 213 in FIG. 2.

Quantification system 200 may be used to associate first read pair 602 with first isoform 102 based on the consistency between first read pair 602 and first isoform 102. For example, splice junction 616 and splice junction 618 of first read pair 602 match isoform splice junction 116 and isoform splice junction 118, respectively, of first isoform 102. Further, contiguously aligned region 608, contiguously aligned region 610, contiguously aligned region 612, and contiguously aligned region 614 are overlapped by the exons of first isoform 102.

Quantification system 200 may be used to associate second read pair 604 with second isoform 104 based on the structural consistency between second read pair 604 and second isoform 104. For example, splice junction 626 aligns with isoform splice junction 120 of second isoform 104. Further, the contiguously aligned regions of third read pair 606 are fully overlapped by the exons of second isoform 104.

Quantification system 200 may determine that third read pair 606 is not consistent with either first isoform 102 or second isoform 104. For example, splice junction 636 and splice junction 638 generally align with isoform splice junction 116 and isoform splice junction 118, respectively, of first isoform 102. However, contiguously aligned region 634 is not fully overlapped by the exons of first isoform 102. Instead, contiguously aligned region 634 overlaps with at least a portion of intron 112 in transcript 100. Accordingly, third read pair 606 is structurally inconsistent with both first isoform 102 and second isoform 104.

II.E. Isoform-Specific and Mutation-Centric Quantification of RNA Expression

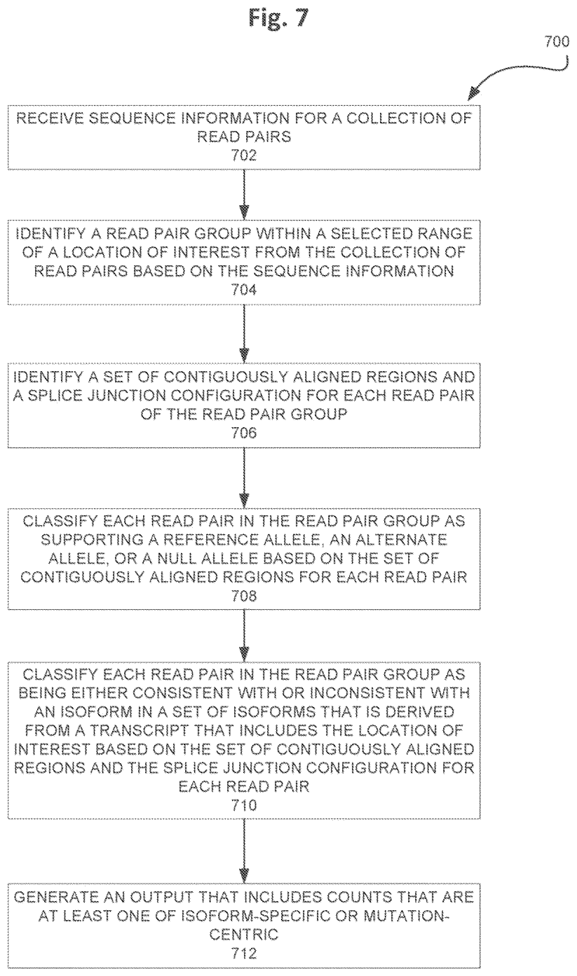

FIG. 7 is a flow diagram of a process for quantifying RNA mutation expression in accordance with one or more embodiments. Process 700 is one example of a process that may be implemented using quantification system 200 or at least a portion of quantification system 200 in FIG. 2. For example, process 700 may be implemented using quantifier 206 in FIG. 2. In some embodiments, process 700 may be implemented using allele classifier 208, isoform analyzer 210, or both in FIG. 2. In various embodiments, at least a portion of the steps in process 700 may be implemented using or in a manner similar to at least a portion of process 300 in FIG. 3, at least a portion of process 400 in FIG. 4, at least a portion of process 500 in FIG. 5, or a combination thereof.

Step 702 includes receiving sequence information for a collection of read pairs. Each read pair in the collection of pairs may be a paired-end read. The collection of read pairs may have been generated from a biological sample using one or more different sequencing technologies. The biological sample may be, for example, a sample extracted from unhealthy tissue, a sample of tumor tissue, a sample of cancerous cells, a sample from a convalescent subject, a sample from a vaccinated subject, or some other type of subject. Read pairs 213 in FIG. 2 may be one example of an implementation for the collection of read pairs in step 702.

Step 704 includes identifying a read pair group within a selected range of a location of interest from the collection of read pairs based on the sequence information. Step 704 may be performed in a manner similar to that described with respect to step 302 in FIG. 3 and step 502 in FIG. 5. The location of interest may be, for example, a location at which a selected mutation (e.g., neoantigen mutation) is expected.

Stop 706 includes identifying a set of contiguously aligned regions and a splice junction configuration for each read pair of the read pair group. Step 706 may be performed in a manner similar to that described with respect to step 304 in FIG. 3 and step 504 in FIG. 5.

Step 708 includes classifying each read pair in the read pair group as supporting a reference allele, an alternate allele, or a null allele based on the set of contiguously aligned regions for each read pair. Stop 706 may include, for example, determining that the read pair supports the reference allele if a nucleotide configuration at the location of interest matches the reference genome at the location of interest. Step 708 may include, for example, determining that the read pair supports the alternate allele if a nucleotide configuration at the location of interest matches the mutation expected at the location of interest. Step 708 may include, for example, determining that the read pair supports a null allele if the nucleotide configuration at the location of interest matches neither the reference genome nor the mutation. In various embodiments, step 708 may be performed in a manner similar to that described with respect to step 306 in FIG. 3 and process 400 in FIG. 4.

Step 710 includes classifying each read pair in the read pair group as being either consistent with or inconsistent with an isoform in a set of isoforms that is derived from a transcript that includes the location of interest based on the set of contiguously aligned regions and the splice junction configuration for each read pair. For example, step 710 may include determining whether a read pair is consistent with the isoform. In step 710, a read pair may be associated with an isoform in a manner similar to that described with respect to step 306 in FIG. 5. In various embodiments, step 710 may be performed for a set of isoforms such that each read pair is classified as being either consistent or inconsistent for each isoform in the set of isoforms.

Step 712 includes generating an output that includes counts that are at least one of isoform-specific or mutation-centric. In step 712, the output may include, for example, any number of or combination of counts that provide information about the number read pairs that are associated with each isoform of a set of isoforms, the number of read pairs that support the reference allele, the number of read pairs that support the alternate allele, or a combination thereof.

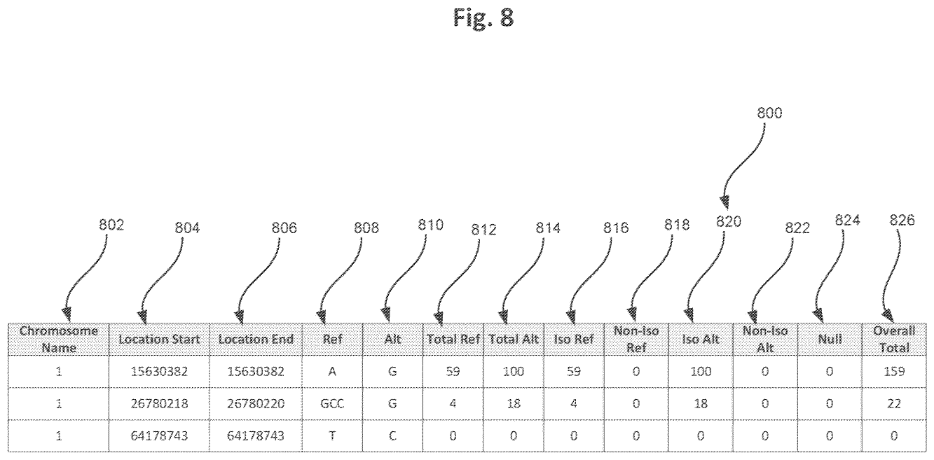

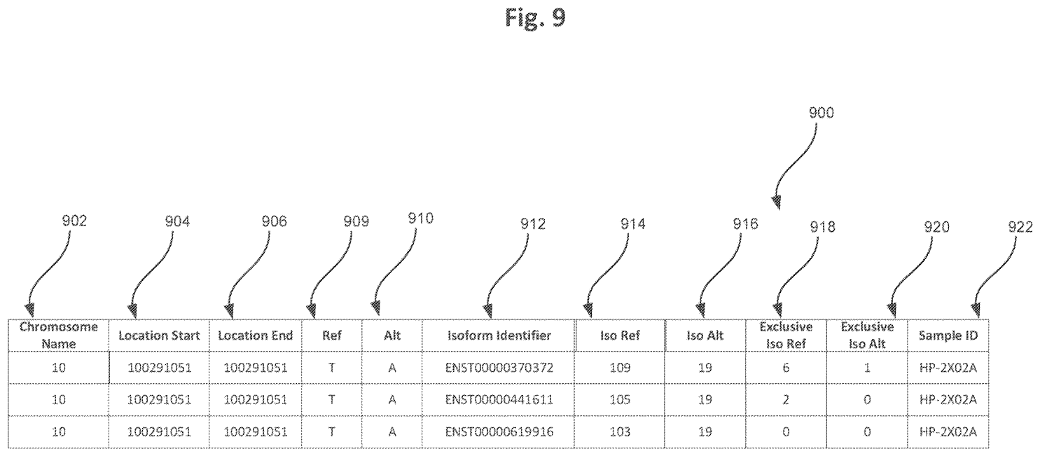

In one or more embodiments, an isoform-specific count is a count of read pairs with respect to a particular isoform. This count may be, for example, without limitation, the number of read pairs consistent with the particular isoform, the number of read pairs consistent with the particular isoform and the reference allele, or the member of read pairs consistent with the particular isoform and the alternate allele. A mutation-centric count is a count of read pairs with respect to a particular mutation. This count may be, for example, without limitation, the number of read pairs that support the alternate allele (e.g., supporting the mutation), the number of read pairs that support the alternate allele and at least one isoform of a set of isoforms (e.g., a set of isoforms that are putatively neoantigen isoforms), or the number of read pairs that support the alternate allele and no isoform of the set of isoforms. Examples of the different types of counts that can be generated are described below with respect to FIGS. 8 and 9.

FIG. 8 is an example of at least a portion of a mutation-centric output in accordance with one or more embodiments. Mutation-centric output 800 is one example of an implementation for mutation-centric output 246 in FIG. 2. Further mutation-centric output 800 may be one example of the mutation-centric output generated in step 308 in FIG. 3 and/or at least a portion of the output generated in step 712 in FIG. 7. In one or more embodiments, mutation-centric output 800 takes the form of a table, a spreadsheet, a file, vector of data, or some other format. In FIG. 8, mutation-centric output 800 is generated for three different mutations (or variants).

Mutation-centric output 800 may identify various types of information including, for example, without limitation, chromosome name 802, location start 804, location end 806, reference allele 808, alternate allele 810, total reference 812, total alternate 814, isoform reference 816, non-isoform reference 818, isoform alternate 820, non-isoform alternate 822, null 824, and overall total 826. Chromosome name 802 may be the name of or other identifier for the chromosome with which the mutation is associated.

Location start 804 and location end 806 together provide the genomic coordinates for the start and end of the location of interest for the mutation. The location of interest may be one or more nucleotides long. Accordingly, location start 804 and location end 806 may identify a same nucleotide position or may span multiple nucleotides.

Reference allele 808 identifies the nucleotide configuration at the location of interest (e.g., as defined location start 804 and location end 806) in the reference genome, without mutation. Alternate allele 810 is the nucleotide configuration of the mutation at the location of interest. The mutation may be an insertion, a deletion, a substitution, or some other type of mutation.

Total reference 812 is a count that identifies the total number of read pairs from a selected read pair group (e.g., read pair group 224 in FIG. 2) classified as supporting the reference allele. Total alternate 814 is a count that identifies the total number of read pairs from the selected read pair group classified as supporting the alternate allele.

Isoform reference 816 is a count that identifies the number of read pairs from the selected read pair group classified as supporting the reference allele and at least one isoform of the set of isoforms associated with the transcript that includes the location of interest. Non-isoform reference 818 is a count that identifies the number of read pairs from the selected read pair group classified as supporting the reference allele and no isoforms of the set of isoforms.

Isoform alternate 820 is a count that identifies the number of read pairs from the selected read pair group classified as supporting the alternate allele and at least one isoform of the set of isoforms. Non-isoform alternate 822 is a count that identifies the number of read pairs from the selected read pair group classified as supporting the alternate allele and no isoforms of the set of isoforms.

Null 824 is a count that identifies the number of read pairs from the selected read pair group classified as supporting neither the reference allele nor the alternate allele. Overall total 826 is a count that identifies the total number of read pairs in the selected read pair group that was processed. The value for overall total 826 may be equal to the sum of total reference 812, total alternate 814, and null 824.

FIG. 9 is an example of at least a portion of an isoform-specific output in accordance with one or more embodiments. Isoform-specific output 900 is one example of an implementation for isoform-specific output 248 in FIG. 2. Further, isoform-specific output 900 may be one example of the isoform-specific output that can be generated in step 508 in FIG. 5 and/or at least a portion of the output generated in step 712 in FIG. 7. In one or more embodiments, isoform-specific output 900 takes the form of a table, a spreadsheet, a file, or some other format. In FIG. 9, isoform-specific output 900 is generated for three different isoforms.

Isoform-specific output 900 may include, for example, without limitation chromosome name 902, location start 904, location end 906, reference allele 908, alternate allele 910, isoform identifier 912, isoform reference 914, isoform alternate 916, exclusive isoform reference 918, exclusive isoform alternate 920, and sample identifier 922. Chromosome name 902 may be the name of or other identifier for the chromosome with which the mutation is associated. Sample identifier 922 identifies the sample from which the read pairs were obtained or generated.

Location start 904 and location end 906 together provide the genomic coordinates for the start and end of the location of interest for the mutation. The location of interest may be one or more nucleotides long. Accordingly, location start 904 and location end 906 may identify a same nucleotide position or may span multiple nucleotides.

Reference allele 908 identifies the nucleotide configuration at the location of interest (e.g., as defined by location start 904 and location end 906) in the reference genome, without mutation. Alternate allele 910 is the nucleotide configuration of the mutation at the location of interest. The mutation may be an insertion, a deletion, a substitution, or some other type of mutation.

Isoform identifier 912 provides the identifier of a specific isoform. Isoform reference 914 is a count that identifies the number of read pairs from a selected read pair group (e.g., read pair group 224 in FIG. 2) classified as supporting the reference allele and consistent with the specific isoform identified by isoform identifier 912. Isoform alternate 916 is a count that identifies the number of read pairs from the selected read pair group classified as supporting the alternate allele and consistent with the specific isoform identified by isoform identifier 912.

Exclusive isoform reference 918 is a count that identifies the number of read pairs from the selected read pair group classified as supporting the reference allele and being exclusively consistent with the specific isoform identified by isoform identifier 912. Exclusive isoform reference 918 is a count that identifies the number of read pairs from the selected read pair group classified as supporting the alternate allele and being exclusively consistent with the specific isoform identified by isoform identifier 912.

II.F. Example Read Pair Analysis

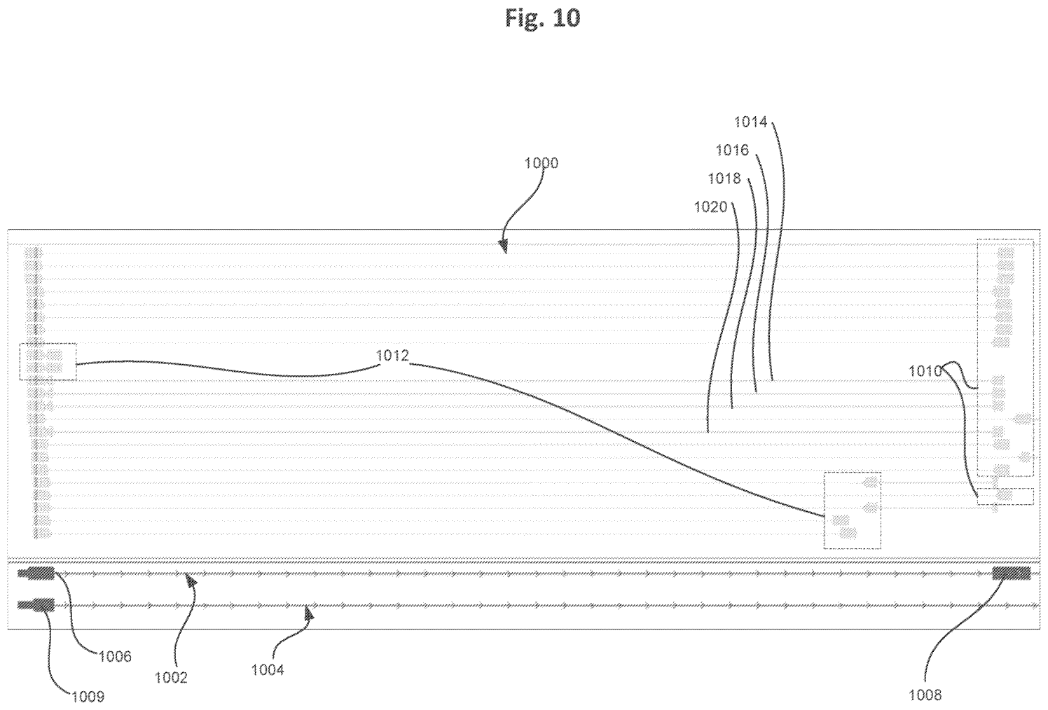

FIG. 10 is a schematic diagram illustrating a read pair group in association with two isoforms in accordance with one or more embodiments. The systems and methods described herein may be used to analyze read group 1000 and quantify RNA expression of read pair group 1000. For example, quantification system 200 in FIG. 2, the processes 300, 400, 500, and/or 700 in FIGS. 3, 4, 5, and 7, respectively, or a combination thereof may be used to quantify RNA expression of a selected mutation in read pair group 1000.

Read pair group 1000 may be one example of at least a portion of read pairs 213 described with respect to FIG. 2. Read pair group 1000 may be one example of read pair group 224 in FIG. 2. Read pair group 1000 is derived from a diseased sample from a subject. Read pair group 1000 may be analyzed with respect to first, isoform 1002 and second isoform 1004 to quantify RNA expression of a selected mutation (e.g., a neoantigen mutation). This quantification enables development of a patient-specific treatment that is designed based on the RNA mutation expression observed in the diseased sample.

First isoform 1002 includes exon 1006 and exon 1008. Second isoform 1004 includes exon 1009. In some embodiments, first isoform 1002 and second isoform 1004 may be two isoforms out of a set of four isoforms that are possible given a particular transcript. Translation of first isoform 1002 and second isoform 1004 may result in different peptides (e.g., neoantigens) being produced, but these two isoforms may have the same mutation.

Read pair group 1000 includes 23 read pairs. From read par group 1000, various sets of read pairs may be identified including, for example, first set of read pairs 1010 and second set of read pairs 1012. First set of read pairs 1010 includes any read pairs that are consistent with at least first isoform 1002 at least because each read pair in first set of read pairs 1010 includes a contiguously aligned region that generally aligns with exon 1008. First set of read pairs 1010 includes 17 read pairs in FIG. 10. Further, first set of read pairs 1010 includes an exclusive set of read pairs that is exclusively consistent with first isoform 1002. The exclusive set of read pairs includes read pair 1014, 1016, 1018, and 1020, each of which includes a splice junction that is unique to first isoform 1002.

Second set of read pairs 1012 includes read pairs that are not consistent with first isoform 1002 or second isoform 1004. Second set of read pairs 1012 includes 6 read pairs in FIG. 10 that include a contiguously aligned region that overlaps with the introns of first isoform 1002 and/or second isoform 1004 and are therefore not consistent with first isoform 1002 or second isoform 1004. In this example in FIG. 10, read pair group 1000 does not include any read pairs that are consistent with second isoform 1004.

Quantification system 200 in FIG. 2 enables generating output 244 for read pair group 1000, which may include mutation-centric output 246, isoform-specific output 248, or both, that provides information (e.g., counts) about the numbers of read pairs in the various sets of read pairs described above. Quantification system 200 enables quantifying the RNA expression of selected mutations in the form of insertions deletions that might otherwise be eliminated from counting using some currently available methods and systems.

Output 244 generated for read pair group 1000 includes one or more isoform-specific counts, one or more of mutation-centric outputs, or both. Output 244 enables determining whether first isoform 1002, second isoform 1004, or both have RNA expression at a level that would make a peptide derived from one of these isoforms a good candidate for treatment development. For example, first set of read pairs 1010 includes 17 read pairs that are consistent with first isoform 1002 but no read pairs of read pair group 1000 are found to be consistent with second isoform 1004. Further, output 244 may indicate that of the 17 read pairs included in first set of read pairs 1010, 15 read pairs support the alternate allele (e.g., have a set of nucleotides that match the selected mutation). These

Accordingly, the peptide derived from second isoform 1004 would make a poor candidate for use in developing a patient-specific treatment as compared to the peptide derived from first isoform 1002. The peptide derived from first isoform 1002, however, would make a good candidate. Thus, a determination may be made to exclude the peptide derived from second isoform 1004 as a target for the patient-specific therapy (e.g., immunotherapy) and to include the peptide derived from first isoform 1002 as the target.

### III. Decision-Making Based on Quantification RNA Mutation Expression

The information provided by the methods and systems described herein (e.g., quantification system 200 in FIG. 2, process 300 in FIG. 3, process 400 in FIG. 4, process 500 in FIG. 5, process 700 in FIG. 7) can be used to make various types of decisions with respect to at least one of treating or predicting the progression or outcome of a disease such as a tumor or cancer. In one or more embodiments, these processes provide a way of quantifying neoantigen mutation expression with respect to specific isoforms. The information generated by this type of quantification may be used to, for example, develop and/or customize neoantigen therapies, such as, for example, neoantigen vaccines.

Neoantigen vaccines can prime a subject's T cells to recognize and attack cancer cells expressing one or more particular tumor neoantigens. This approach can generate a tumor-specific immune response that spares healthy cells while targeting tumor cells. The individualized vaccine may be engineered or selected based the information generated by the various embodiments described above.

An immunotherapy such as, for example, without limitation, a cancer treatment may include collecting a sample (e.g., a blood sample) from a subject. T cells can be isolated and stimulated. The isolation can be performed using, for example, density gradient sedimentation (e.g., and centrifugation), immunomagnetic selection, and/or antibody-complex filtering. The stimulation may include, for example, antigen-independent stimulation, which may use a mitogen (e.g. PHA or Con A) or anti-CD3 antibodies (e.g., to bind to CD3 and activate the T-cell receptor complex) and anti-CD28 antibodies (e.g., to bind to CD28 and stimulate T cells). A set of peptides (e.g., mutant peptides) can be selected to use in the treatment of the subject based on the information provided by the various embodiments described above corresponding to which neoantigen mutation expression at levels that would indicate an immune response would be triggered in the subject.

In some embodiments, the set of peptides (or precursors thereof) can be used to produce mutant peptide (for example, neoantigen) specific T cells. For example, peripheral blood T cells can be isolated from a subject and contacted with one or more mutant peptides to induce mutant peptide-specific T-cells populations that can be administered to a subject. In some examples, the T cell receptor sequence of the mutant peptide-reactive T cells can be sequenced. Once the T-cell receptor sequence (e.g., amino acid T-cell receptor sequence) is obtained. T cells can be engineered to include the T cell receptor that specifically recognizes the mutant peptide. These engineered T cells can then be administered to a subject. See, e.g., Matsuda et al. “Induction of Neoantigen-Specific Cytotoxic T Cells and Construction of T-cell Receptor Engineered T Cells for Ovarian Cancer,” Clin. Cancer Res. 1-11 (2018), hereby incorporated by reference in its entirety for all purposes. The T cells can be expanded in vitro and/or ex vivo prior to administration to a subject. The subject may then be administered (e.g., infused with) a position that includes the expanded population of T cells. In one or more embodiments, the treatment is administered to an individual in an amount effective to, for example, prime, activate and expand T cells in vivo.

Thus, the embodiments described herein may provide information that may be important to the selection of neoantigens for use in generating neoantigen therapies Quantification of isoform-specific neoantigen mutation expression may allow or enable deprioritizing neoantigens derived from mutated isoforms with little to no RNA expression, filtering out non-expressed neoantigen mutations, investigation of the determinants of expression, or a combination thereof.

For example, output 244 in FIG. 2, the mutation-centric output generated in step 308 in FIG. 3, the isoform-specific output generated in step 508 in FIG. 5, the output generated in step 712 in FIG. 7 may be used to determine whether to include or exclude the antigens derived from different isoforms. For example, a determination may be made to include an antigen (e.g., neoantigen) that is derived from a particular isoform having the selected mutation as a target for an immunotherapy in response to one or more of these outputs indicating at least a threshold level of RNA expression for the selected mutation, at least a threshold level of RNA expression for the particular isoform, or both. Alternatively, a determination may be made to exclude an antigen as a target for an immunotherapy in response to one or more of these outputs indicating that at least one of the RNA expression for the selected mutation, the RNA expression for the particular isoform, or both are below the threshold level.

The threshold level of RNA expression may include, for example, a threshold count of read pairs that are consistent with the particular isoform. This threshold count may be, for example, 5, 8, 10, 15, 20, 25, 50, 100, 200, 300, 500, 1000, 2000, or some other number of read pairs. In some cases, the threshold level of RNA expression for the selected mutation may be different from the threshold level of RNA expression for the particular isoform. The immunotherapy may include, for example, without limitation, at least one of a T cell therapy, a personalized cancer therapy, a cancer immunotherapy, an antigen-specific immunotherapy, an antigen-dependent immunotherapy, a vaccine, a natural killer (NK) cell therapy, or some other type of customized therapy.

Output 244 in FIG. 2, the mutation-centric output generated in step 308 in FIG. 3, the isoform-specific output generated in step 508 in FIG. 5, or the output generated in step 712 in FIG. 7 may provide an indication of subject-specific (e.g., patient-specific) RNA expression for a set of peptides based on a diseased sample. These outputs may be used to design and/or manufacture a treatment that includes at least one of a peptide from the set of peptides, a precursor of the peptide, nucleic acids that encode the peptide, or a plurality of cells that express the peptide. In some cases, mRNA that code for at least one peptide of the set of peptides may be synthesized and then complexed with lipids to produce mRNA-lipoplex. The mRNA-lipoplex may then be administered to the subject.

Further, output 244 in FIG. 2, the on-centric output generated in step 308 in FIG. 3, the isoform-specific output generated in step 508 in FIG. 5, or the output generated in step 712 in FIG. 7 may be used to produce a vaccine that comprises one or more peptides; a plurality of nucleic acids that encode the one or more peptides; or a plurality of cells expressing the one or more peptides.

### IV. Computer-Implemented System

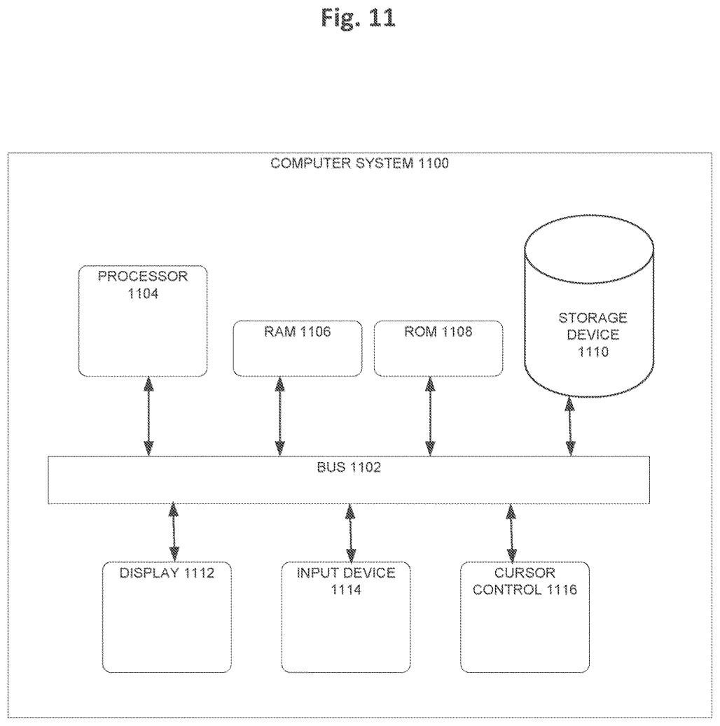

FIG. 11 is a block diagram illustrating an example of a computer system in accordance with various embodiments. Computer system 1100 may be an example of one implementation for computer system 202 described above in FIG. 2. In one or more examples, computer system 1100 can include a bus 1102 or other communication mechanism for communicating information, and a processor 1104 coupled with bus 1102 for processing information. In various embodiments, computer system 1100 can also include memory, which can be a random-access memory (RAM) 1106 or other dynamic storage device, coupled to bus 1102 for determining instructions to be executed by processor 1104. Memory also can be used for storing temporary variables or other intermediate information during execution of instructions to be executed by processor 1104. In various embodiments, computer system 1100 can further include a read-only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device, is a cursor control 1116, such as a mouse, a joystick, a trackball, gesture-input device, a gaze-based input device, or cursor direction keys for communicating direction information and command selections to processor 1104 and for controlling cursor movement on display 1112. This input device 1114 typically has two degrees of freedom in two axes, a first axis (e.g., x) and a second axis (e.g., y), that allows the device to specify positions in a plane. However, it should be understood that input devices 1114 that allow for three-dimensional (e.g., x, y and z) cursor movement are also contemplated herein.

Consistent with certain implementations of the present teaching, results can be provided by computer system 1100 in response to processor 1104 executing one or more sequences of one or more instructions contained in RAM 1106. Such instructions can be read into RAM 1106 from another computer-readable medium of computer-readable storage medium, such as storage device 1110. Execution of the sequences of instructions contained in RAM 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, magnetic disks, such as storage device 1110. Examples of volatile media can include, but are not limited to, dynamic memory, such as RAM 1106. Examples of transmission media can include, but are not limited to, coaxial cables, copper wire, and fiber optics, including the wires that comprise bus 1102.

Common forms of computer-readable media, include, for example, a floppy disk, a flexible disk, hard disk, magnetic tape, any other ma medium, a CD-ROM, any other optical medium, punch cards, paper tape, any other physical medium with patterns of holes, a RAM, PROM, and EPROM, a FLASH-EPROM, soy her memory chip or cartridge, or any other tangible medium from which a computer can read.

In addition to computer readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1100 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital processors (DSPs), digital signal devices (DSPDs), programmable logic-devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, the memory components RAM 1106, ROM, 1108, or storage device 1110 and user input provided via input device 1114.

### V. Exemplary Context and Definitions

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” may be 2, 3, 4, 5, 6, 7, 8, 9, 10, or more.

As used herein, the term “set of” may be one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be used. For example, without limitation, “at least one of item A, item B, or item C” means item A, item A and item B; item B; item A, item B, and item C; item B and item C, or item A and C. In some cases, “least one of item A, item B, or item C” means, but is not limited to, two of item A, one of item B, and ten of item C; four of item B and seven of item C; or some other suitable combination. Where reference is made to a list of elements (e.g., elements a, b, c), such reference is intended to include any one of the listed elements by itself, any combination of less than all of the listed elements, and/or a combination of all of the listed elements.

As used herein, a “subject” may refer to a mammal being assessed for treatment and/or being treated, a mammal participating in a clinical trial, a mammal undergoing anti-cancer therapies, or any other mammal of interest. In various embodiments, the terms “subject,” “individual,” and “patient” are used interchangeably herein. A subject can be a healthy or asymptomatic individual, an individual that has or is suspected of having a disease (e.g., cancer) or pre-disposition to the disease, an individual that is in need of therapy or suspected of needing therapy, or a combination thereof. A subject may be, for example, without limitation, an individual having cancer or an individual having an autoimmune disease. A subject may be human. In other cases, a subject may be some other type of mammal. For example, a subject may be a mammal used in forming laboratory models for human disease. Such mammals include, but are not limited to, mice, rats, primates (e.g., cynomolgus monkey), etc.

As used herein, a “sample” may refer to “biological sample” of a subject. A sample can include tissue (e.g., a biopsy), single cell, multiple cells, fragments of cells or an aliquot of body fluid. The sample may have taken from a subject, by means including venipuncture, excretion, ejaculation, massage, biopsy, needle aspirate, lavage sample, scraping, surgical incision, or intervention or other means known in the art.

As used herein, a “nucleotide,” may comprise a nucleotide and a phosphate group. A “nucleotide,” as used herein, comprises a nucleobase and a five-carbon sugar (e.g., ribose, deoxyribose, or analog thereof). When the nucleobase is bonded to ribose, the nucleotide may be referred to as a ribonucleoside. When the nucleobase is bonded to deoxyribose, the nucleotide may be referred to as a deoxyribonucleoside. A “nucleobase,” which may be also referred to as a “nitrogenous base,” can take the form of one of five types: adenine (A), guanine (G), thymine (T), uracil (U), and cytosine (C).

As used herein, a “polynucleotide,” “nucleic acid,” or “oligonucleotide” may refer to a linear polymer of nucleotides (or nucleosides joined by internucleosidic linkages). Generally, a polynucleotide comprises at least three nucleotides. Generally, an oligonucleotide is comprised of nucleotides that range in number from a few nucleotides (or monomeric units) to several hundreds of nucleotides (monomeric units). Whenever a polynucleotide such as an oligonucleotide is represented a sequence of letters, such as “ATGCCTG,” it will be understood that the nucleotides are in 5′→3′ order or direction from left to right and that “A” denotes adenine, “C” cytosine, “G” denotes guanine, and “T” denotes thymine, unless otherwise noted. The letters A, C, G, and T may be used to refer to the nucleobases themselves, as described above, the nucleotides that include those nucleobases, or the nucleotides that include those bases, as is standard in the art.

Deoxyribonucleic acid (DNA) is a chain of nucleotides consisting of 4 types of nucleotides: adenine (A), thymine (T), cytosine (C), and guanine (G). Ribonucleic acid (RNA) is comprised of 4 types of nucleotides: A, C, G, and uracil (U). Certain pairs of nucleotides specifically bind to one another in a complementary fashion, which may be referred to as complementary base pairing. For example, C pairs with G and A pairs with T. In the case of RNA, however, A pairs with U. When a first nucleic acid strand binds to a second nucleic acid strand made up of nucleotides that are complementary to those in the first strand, the two strands bind to form a double strand.

As used herein, “nucleic acid sequencing data,” “nucleic acid sequencing information,” “nucleic acid sequence,” “genomic sequence,” “genetic sequence,” “fragment sequence,” “nucleic acid sequencing read” denotes any information or data that is indicative of the order of the nucleotide bases (e.g., A, C, G, T/U) in a molecule (e.g., whole genome, whole transcriptome, exome, oligonucleotide, polynucleotide, fragment, etc.) of DNA or RNA. It should be understood that the present disclosure contemplates that this sequence information may be obtained using any of the available varieties of techniques, platforms, or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic-based Systems, etc., or a combination thereof.

A term “genome,” as used herein, may refer to the genetic material of a cell or organism, including animals, such as mammals (e.g., humans), and comprises nucleic acids, such as DNA. A genome is stored on one or more chromosomes comprised of DNA sequences. In humans, DNA includes, for example, genes, noncoding DNA, and mitochondrial DNA. The human genome typically contains 23 pairs of chromosomes: 22 pairs of autosomal chromosomes (autosomes) plus the sex-determining X and Y chromosomes. The 23 pairs of chromosomes include one copy from each parent. The DNA that makes up the chromosomes is referred to as chromosomal DNA and is present in the nucleus of human cells (nuclear DNA).

As used herein, a “gene” may be a discrete portion of heritable, genomic sequence which affect a subject's traits by being expressed as a functional product or by regulation of gene expression. The total complement of genes in a subject or cell is known as the subject's or cell's genome. A region of a chromosome at which a particular gene is located is called its locus. Each locus contains one allele of a gene. Thus, a pair of chromosomes together has two loci that each contain an allele of the gene to form an allele pair. The two alleles may be the same or may be different (e.g., have slightly varying gene sequences).

As used herein, an “allele” may be a variant of a particular a nucleotide configuration at a location of interest. The nucleotide configuration may be comprised of, for example, one or more nucleotides.

As used herein, a “sequence” may denote any information or data that is indicative of the order of the nucleotide bases (e.g. A, C, G, T/U) in a molecule (e.g., whole genome, whole transcriptome, exome, oligonucleotide, polynucleotide, fragment, etc.) of DNA or RNA. Sequence information may be obtained using any of the available varieties of techniques, platforms, or technologies, including, but not limited to, capillary electrophoresis, microarrays, ligation-based systems, polymerase-based systems, hybridization-based stems, direct or indirect nucleotide identification systems, pyrosequencing, ion- or pH-based detection systems, electronic-based systems, etc., or a combination thereof. As one example, sequence information may be obtained using next generation sequencing.

As used herein, “next generation sequencing” (NGS) may refer to sequencing technologies having increased throughput as compared to traditional Sanger- and capillary electrophoresis-based approaches. These sequencing technologies have, for example, the ability to generate hundreds of thousands of relatively small sequence reads or “reads” at a time. Some examples of next generation sequencing techniques include, but are not limited to, sequencing by synthesis, sequencing by ligation, and sequencing by hybridization.

As used herein, a “read” or “sequence read” may include a string of nucleic acid bases corresponding to a nucleic acid molecule that has been sequenced. For example, a read can refer to the sequence of nucleotides determined for a nucleic acid fragment that has been subjected to sequencing, such as, for example, next generation sequencing (“NGS”) Reads can be any sequence of any number of nucleotides, with the number of nucleotides defining the read length.

As used herein, a “T cell”, also known as a T lymphocyte, may refer to a type of an adaptive immune cell. T cells develop in the thymus gland and play a central role in the immune response of the body. T cells can be distinguished from other lymphocytes by the presence of a T cell receptor (TCR) on the cell surface. These immune cells originate as precursor cells, derived from bone marrow; and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. Engineered T cells may be designed to recognize MHC-presented peptides. For example, an engineered T cell may be designed with an antigen that is not subject to HLA loss. Engineered T cells can be formed by the millions or billions in the laboratory and then infused into a patient's body. Engineered T cells may be designed to multiply and recognize the cancer cells that express a specific protein or neoantigen. This type of technology may be used in potential next-generation immunotherapy treatment.

As used herein, “immunotherapy” may refer to a treatment or class of treatments that uses one or more parts of a subject's immune system to fight a disease such as, for example, without limitation, cancer. Immunotherapy can use substances made by the body or synthesized outside of the body to improve bow the immune system works to find and destroy cancer cells.

As used herein, the terms “peptide”, “polypeptide” and “protein” are used interchangeably to refer to a polymer of amino acid residues. The terms encompass amino acid chains of any length, including full-length proteins with amino acid residues linked by covalent peptide bonds.

As used herein, a “mutant peptide” may refer to a peptide resent in the wild type amino acid sequences of normal tissue of an individual subject. A mutant peptide may comprise at least one mutant amino acid present in a disease tissue (e.g., collected from a particular subject) but not in a normal tissue (e.g., collected from the particular subject, collected from a different subject and/or as identified in a database as corresponding to normal tissue). A mutant peptide may include an epitope and thus is a substance that induces an immune response (as a result of not being associated with a subject's “self”). A mutant peptide can include and/or can be a neoantigen. A mutant peptide can arise from, for example: a non-synonymous mutation leading to different amino acids in the protein (e.g., point mutation); a read-through mutation in which a stop codon is modified or deleted; leading to translation of a longer protein with a novel tumor-specific sequence at the C-terminus; a splice site mutation that leads to a unique tumor-specific protein sequence; a chromosomal rearrangement that gives rise to a chimeric protein with a tumor-specific sequence at a junction of two proteins (i.e., gene fusion) and/or a frameshift insertion or deletion that leads to a new open reading frame with a tumor-specific protein sequence. A mutant peptide can include a polypeptide (as characterized by a polypeptide sequence) and/or may be encoded by a nucleotide sequence.

As used herein, a “neoantigen” may refer be a tumor-specific antigen derived from somatic mutations in tumors and presented by a subject's cancer cells and a antigen presenting cells. Neoantigen therapies, such as, but not limited to, neoantigen vaccines, are a relatively new approach for providing individualized cancer treatment. Neoantigen vaccines can prime a subject's T cells to recognize and attack cancer cells expressing or more particular tumor neoantigens. This approach generates a tumor-specific immune response that spares healthy cells while targeting tumor cells. The individualized vaccine may be engineered or selected based on a subject-specific tumor profile. The tumor profile can be defined by determining DNA and/or RNA sequences from a subject's tumor cell and using the sequences to identify neoantigens that are present in tumor cells but absent in normal cells.

As used herein, a Compact Idiosyncratic Gapped Alignment Report (CIGAR) string may be one format for representing a read or read pair with respect to alignment to a reference genome. A CIGAR string is typically associated with a position that denotes the leftmost coordinate (e.g., nucleotide position) of alignment of a particular sequence to the reference genome. A CIGAR string include various operations such as, but not limited to, an “M” for a match that indicates an exact match of x positions between the sequence and the reference genome; an “N” for an alignment gap that indicates that the next x positions of the reference genome do not match the sequence; a “D” for a deletion that indicates that the next x positions of the reference genome do not match the sequence; and an “I” for an insertion that indicates that the next x positions of the sequence do not match the reference genome. For example, a CIGAR string of “3M2I2M1D2M” indicates 3 matches, 2 insertions, 2 matches, 1 deletion, and 2 matches.

As used herein, “immunogenic” refers to the ability to elicit an immune response (e.g., via T cells and/or B cells).

### VI. Additional Considerations

The headers and subheaders between sections and subsections of this document are included solely for improving readability and do not imply that features cannot be combined across sections and subsection. Accordingly, sections and subsections do not describe separate embodiments.

Some embodiments of the present disclosure include a system including one or more data processors. In some embodiment the system includes a non-transitory computer readable storage medium containing instructions which, when executed on one of more data processors, cause the one or more data processors to, perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, although the present invention as claimed has been specifically disclosed by embodiments and optional features, it should be understood that modification and variation of the concepts disclosed herein may be employed by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

### VII. Embodiments

Various embodiments may include:
Embodiment 1: A computer-implemented method for quantifying ribonucleic acid (RNA) mutation expression, the computer-implemented method comprising: identifying, for each read pair in a read pair group, a set of contiguously aligned regions and a splice junction configuration, wherein each read pair is within a selected range of a location of interest; classifying each read pair in the read pair group based on the set of contiguously aligned regions and the splice junction configuration that correspond to each read pair, a reference genome, send a selected mutation; and generating a mutation-centric output for the read pair group.
Embodiment 2: The computer-implemented method of embodiment 1, wherein the splice junction configuration for a read pair in the read pair group identifies a presence of a splice junction in the read pair.
Embodiment 3: The computer-implemented method of any one of embodiments 1 or 2, wherein the selected mutation is a mutation comprising a set of nucleotides, the mutation having been previously identified as occurring at the location of interest in a genome derived from a diseased sample.
Embodiment 4: The computer-implemented method of any one of embodiments 1-3, wherein the classifying a read pair in the read pair group comprises: classifying the read pair as supporting a reference allele when an allele at the location of interest mate the reference genome at the location of interest.
Embodiment 5: The computer implemented method of any one of embodiments 1-4, wherein classifying each read pair in the read pair group comprises: classifying the read pair as supporting an alternate allele when an allele at the location of interest matches the selected mutation at the location of interest.
Embodiment 6: The computer-implemented method of any one of embodiments 1-5, wherein classifying each read pair in the read pair group comprises: classifying the read pair as supporting a null allele when an allele at the location of interest does not match either the reference genome or the selected mutation at the location of interest.
Embodiment 7: The computer-implemented method of any one of embodiments 1-6, wherein the select mutation is a neoantigen mutation.
Embodiment 8: The computer-implemented method of any one of embodiments 1-7, further comprising: receiving sequence information for a plurality of read pairs; and identifying a portion of the plurality of read pairs that fall within the selected range of the location of interest based on the sequence information to form the read pair group.
Embodiment 9: The computer-implemented method of any one of embodiments 1-8, wherein the mutation is an indel and wherein classifying each read pair in the read pair group comprises: identifying an alignment gap between two contiguously aligned regions within the read pair at the location of interest, wherein the alignment gap comprises at least one nucleotide that does not align with the reference genome and that is flanked by the two contiguously aligned regions.
Embodiment 10: The computer-implemented method of embodiment 9, wherein the classifying further comprises: classifying the read pair as supporting an alternate allele when the at least one nucleotide matches the selected mutation.
Embodiment 11: The computer implemented method of any one of embodiments 1-8, wherein the selected mutation is a single nucleotide variation (SNV) and wherein classifying each read pair in the read pair group comprises: classifying the allele at the location of interest within the read pair pairs based on a nucleotide at the location of interest, wherein the allele is classified as a reference allele if the nucleotide matches the reference genome at the location of interest; wherein the allele is classified as an alternate allele if the nucleotide matches the selected mutation at the location of interest; and wherein the allele is classified as a null allele if the nucleotide matches neither the reference genome nor the selected mutation at the location of interest.
Embodiment 12: The computer-implemented method of any one of embodiments 1-8, wherein the selected mutation is a single nucleotide variation (SNV) and wherein classifying each read pair in the read pair group comprises: classifying the read pair as a skip when the location of interest does not fall within a contiguously aligned region within the read pair due to deletion.
Embodiment 13: The computer-implemented method of any one of embodiments 1-12, further comprising: associating a read pair in the read pair group with an isoform derived from a transcript that includes the location of interest.
Embodiment 14: The computer-implemented method of any one of embodiments 1-13, further comprising: associating a read pair in the read pair group with an isoform derived from a transcript that includes the location of interest based on the set of contiguously aligned regions within the read pair and the splice junction configuration for the read pair.
Embodiment 15: The computer-implemented method of embodiment 14, further comprising: associating a read pair in the read pair group with the isoform derived from a transcript that includes the location of interest when the splice junction configuration of the read pair is consistent with a set of isoform splice junctions within the isoform and the set of contiguously aligned regions within the read pair is overlapped by a set of exons within the isoform.
Embodiment 16: The computer-implemented method of any one of embodiments 1-15, further comprising: associating a read pair in the read pair group with an isoform derived from a transcript that includes the location of interest when the splice junction configuration of the read pair is consistent with a set of isoform splice junctions within the isoform.
Embodiment 17: The computer-implemented method of any one of embodiments 1-16, further comprising: associating a read pair in the read pair group with an isoform derived from a transcript that includes the location of interest when the set of contiguously aligned regions within the read pair is fully overlapped by a set of exons within the isoform.
Embodiment 18: The computer-implemented method of any one of embodiments 1-17, wherein the mutation-centric output comprises a count of read pairs in the read pair group that support a reference allele.
Embodiment 19: The computer-implemented method of any one of embodiments 1-18, wherein the mutation-centric output comprises a count of read pairs in the read pair group that support a null allele.
Embodiment 20: The computer-implemented method of any one of embodiments 1-19, wherein the mutation-centric output comprises a count of read pairs in the read pair group that support a null allele.
Embodiment 21: The computer-implemented method of any one of embodiments 1-20, wherein the mutation-centric output comprises a count of read pairs in the read pair group that support a reference allele and are consistent with at least one isoform derived from a transcript that includes the location of interest.
Embodiment 22: The computer-implemented method of any one of embodiments 1-21, wherein the mutation-centric output comprises a count of read pairs in the read pair group that support an alternate allele and are consistent with at least one isoform derived from a transcript that includes the location of interest.
Embodiment 23: The computer-implemented method of any one of embodiments 1-22, wherein the mutation-centric output comprises a count of read pairs in the read pair group that support a reference allele and are consistent with no isoforms derived from a transcript that includes the location of interest.
Embodiment 24: The computer-implemented method of any one of embodiments 1-23, wherein the mutation-centric output comprises a count of read pairs in the read pair group that support an alternate allele and are consistent with no isoforms derived from a transcript that includes the location of interest.
Embodiment 25: The computer-implemented method of any one of embodiments 1-24, further comprising: determining to include an antigen that is derived from the selected mutation as a target for an immunotherapy responsive to the mutation-centric output indicating at least a threshold level of RNA expression for the selected mutation.
Embodiment 26: The computer-implemented method of any one of embodiments 1-25, further comprising: determining to exclude an antigen that is derived from the selected mutation as a target for an immunotherapy responsive to the mutation-centric output indicating that RNA expression for the selected mutation is below a threshold level.
Embodiment 27: The computer-implemented method of embodiment 25 or embodiment 26, wherein the antigen is a neoantigen.
Embodiment 28: The computer-implemented method of any one of embodiments 25-27, wherein the immunotherapy is a target antigen-specific immunotherapy, optionally wherein the target antigen-specific immunotherapy is a T cell therapy or a personalized cancer vaccine.
Embodiment 29: The computer-implemented method of any one of embodiments 1-28, wherein the read pair group is derived from a diseased sample from a subject.
Embodiment 30: The computer-implemented method of any one of embodiments 1-29, wherein the read pair group is derived from cancer cells from a subject.
Embodiment 31: The computer-implemented method of any one of embodiments 1-30, wherein the mutation-centric indicates RNA expression for the selected mutation and further comprises: determining that the selected mutation has at least a threshold level of RNA expression; and developing a treatment that includes at least one of a peptide that is derived from the selected mutation a precursor of the peptide, a nucleic acid that encodes the peptide, or a plurality of cells that express the peptide.
Embodiment 32: The computer-implemented method of embodiment 31, wherein the peptide is a neoantigen and wherein the treatment is a neoantigen treatment.
Embodiment 33: The computer-implemented method of embodiment 31 or embodiment 32, wherein the read pair group is derived from a disease sample of a subject such that the treatment is personalized for the subject.
Embodiment 34: The computer-implemented method of any one of embodiments 31-33, wherein the treatment is a cancer immunotherapy.
Embodiment 35: The computer-implemented method of any one of embodiments 31-34, wherein the treatment is a vaccine.
Embodiment 36: A computer-implemented method for quantifying isoforms, the computer-implemented method comprising: identifying, for each read pair of a read pair group, a set of contiguously aligned regions and a splice junction configuration, wherein each read pair is within a selected range of a location of interest; evaluating whether each read pair in the read pair group is consistent with or inconsistent with a first isoform that is derived from a transcript that includes the location of interest based on the set of contiguously aligned regions and the splice junction configuration identified for each read pair; and generating an isoform-specific output that identifies a first count for read pairs in the read pair group that is consistent with the first isoform.
Embodiment 37. The computer-implemented method of embodiment 36, wherein evaluating a read pair in the read pair group comprises: determining that the read pair is consistent with the first isoform in response to a determination that the splice junction configuration of the read pair is consistent with a set of isoform splice junctions within the first isoform.
Embodiment 38: The computer-implemented method of embodiment 36 or embodiment 37, wherein evaluating a read pair in the read pair group comprises: determining that the read pair is consistent with the first isoform in response a determination that the set of contiguously aligned regions within the read pair is overlapped by a set of exorts within the first isoform.
Embodiment 39: The computer-implemented method of any one of embodiments 36-38, wherein evaluating a read pair in the read pair group comprises: determining that the read pair is exclusively consistent with the first isoform using at least the splice junction configuration for the read pair.
Embodiment 40: The computer-implemented method of any one of embodiments 36-39, further comprising: evaluating whether each read pair in the read pair group is consistent with or inconsistent with a second isoform that is derived from the transcript that includes the location of interest based on the set of contiguously aligned regions and the splice junction configuration identified for each read pair.
Embodiment 41: The computer-implemented method of embodiment 40, wherein generating the isoform-specific output further comprises identifying at least a second count for read pairs in the read pair group that are consistent with the second isoform.
Embodiment 42: The computer-implemented method of embodiment 41, wherein the second count includes at least one read pair in the read pair group that is also included in the first count.
Embodiment 43: The computer-implemented method of any one of embodiments 36-42, wherein the first count identifies a number of read pairs from the read pair group that are exclusively consistent with the first isoform.
Embodiment 44: The computer-implemented method of any one of embodiments 36-43, wherein the isoform-specific output further identifies a second count that a number of read pairs from the read pair group that are exclusively consistent with the first isoform.
Embodiment 45: The computer-implemented method of any one of embodiments 36-44, further comprising: determining to include an antigen that is derived from the first isoform as a target for an immunotherapy responsive to the isoform-centric output when RNA expression for the first isoform is at least a threshold level.
Embodiment 46: The computer-implemented method of any one of embodiments 36-45, further comprising: determining to exclude an antigen that is derived from the selected mutation as a target for an immunotherapy responsive to the isoform-specific output when RNA expression for the first isoform is below a threshold level.
Embodiment 47: The computer-implemented method of embodiment 45 or embodiment 46, wherein the antigen is a neoantigen.
Embodiment 48: The computer-implemented method of any one of embodiments 45-47, wherein the immunotherapy is a target antigen-specific immunotherapy, optionally wherein the target antigen-specific immunotherapy is a T cell therapy or a personalized cancer vaccine.
Embodiment 49: The computer-implemented method of any one of embodiments 36-48, wherein the read pair group is derived from a diseased sample from a subject.
Embodiment 50: The computer-implemented method of any one of embodiments 36-49, wherein the read pair group is derived from cancer cells from a subject.
Embodiment 51: The computer-implemented method of any one of embodiments 36-50, wherein the isoform-specific output indicates RNA expression for the first isoform and further comprising: determining that the first isoform has at least a threshold level of RNA expression: and developing a treatment that includes at least one of a peptide that is derived from the first isoform, a precursor of the peptide, a nucleic acid that encodes the peptide, or a plurality of cells that express the peptide.
Embodiment 52: The computer-implemented method of embodiment 51, wherein the peptide is a neoantigen and wherein the treatment is a neoantigen treatment.
Embodiment 53: The computer-implemented method of embodiment 51 or embodiment 52, wherein the read pair group is derived from a disease sample of a subject such that the treatment is personalized for the subject.
Embodiment 54: The computer-implemented method of any one of embodiments 51-53, wherein the treatment is a cancer immunotherapy.
Embodiment 55: The computer-implemented method of any one of embodiments 51-54, wherein the treatment is a vaccine.
Embodiment 56: A computer-implemented method for quantifying isoform-specific RNA mutation expression, the computer-implemented method comprising, identifying a set of contiguously aligned regions and a splice junction configuration for each read pair in a read pair group within a selected range of a location of interest at which a selected mutation is expected; classifying each read pair in the read pair group as supporting either a reference allele, an alternate allele, or a null allele based on the set of contiguously aligned regions for each read pair; classifying each read pair in the read pair group as being either consistent with or inconsistent with an isoform in a set of isoforms that is derived from a transcript that includes the location of interest based on the set of contiguously aligned regions and the splice junction configuration for each read pair; and generating an output that includes counts that are at least one of isoform-specific or mutation-centric.
Embodiment 57: The computer-implemented method of embodiment 56, further comprising, receiving sequence information for a collection of read pairs; and identifying the read pair group within the selected mange of the location of interest from the collection of read pairs based on the sequence information.
Embodiment 58 The computer-implemented method of embodiment 56 or embodiment 57, wherein the selected mutation is an indel and wherein classifying a read pair in the read paus group comprises: identifying an alignment gap between two contiguously aligned regions within the read pair at the location of interest, wherein the alignment gap comprises at least one nucleotide that does not align with the reference genome and that is flanked by the two contiguously aligned regions.
Embodiment 59: The computer-implemented method of embodiment 58, wherein classifying a read pair in the read pair group further comprises: classifying the read pair as supporting the alternate allele when the at least one nucleotide matches the selected mutation.
Embodiment 60: The computer-implemented method of embodiment 58 or embodiment 59, wherein classifying a read pair in the read pair group further comprises: classifying the read pair as supporting the null allele when the at least one nucleotides does not match the selected mutation or the reference genome.
Embodiment 61: The computer-implemented method of embodiments 56 or embodiment 57, wherein classifying a read pair in the read pair group further comprises: classifying the read pair as supporting the reference allele when the location of interest matches the reference genome at the location of interest.
Embodiment 62: The computer-implemented method of any one of embodiments 56-61, wherein the selected mutation is a single nucleotide variation (SNV) and wherein classifying a read pair in the read pair group comprises, classifying a read pair in the read pair group based on a single nucleotide at the location of interest, wherein the allele is classified as the reference allele if the nucleotide matches the reference genome at the location of interest; wherein the allele is classified as the alternate allele if the nucleotide matches the selected mutation at the location of interest, and wherein the allele is classified as the null allele if the nucleotide matches neither the reference genome nor the selected mutation at the location of interest.
Embodiment 63: The computer-implemented method of any one of embodiments 56-62, wherein classifying a read pair in the read pair group as being either consistent with or inconsistent with the isoform comprises: classifying the read pair as consistent with the isoform in response to a determination that the splice junction configuration of the read pair is consistent with a set of isoform splice junctions within the isoform.
Embodiment 64: The computer implemented method of any one of embodiments 56-63, wherein classifying a read pair in the read pair group as being either consistent with or inconsistent with the isoform comprises: classifying the read pair consistent with the isoform in response to a determination that the set of contiguously aligned ions within the read pair is fully overlap of exons within the isoform.
Embodiment 65: The computer-implemented method of any one of embodiments 56-64, wherein the output includes a count of read pairs in the read pair group that support the reference allele.
Embodiment 66: The computer-implemented method of any one of embodiments 56-65, wherein the output includes a count of read pairs in the read pair group that support the alternate allele.
Embodiment 67: The computer-implemented method of any one of embodiments 56-66, wherein the output includes a count of read pairs in the read pair group that support the null allele.
Embodiment 68: The computer-implemented method of any one of embodiments 56-67, wherein the output includes a count of read pairs in the read pair group that support the reference allele and are consistent with at least one isoform in the set of isoforms.
Embodiment 69: The computer-implemented method of any one of embodiments 56-68, wherein the output includes a count of read pairs in the read pair group that support alternate allele and are consistent at least one isoform in the set of isoforms.
Embodiment 70: The computer-implemented method of any one of embodiments 56-69, wherein the output includes a count of read pairs in the read pair group that support the reference allele and are consistent with no isoforms in the set of isoforms.
Embodiment 71: The computer-implemented method of any one of embodiments 56-70, wherein the output includes a count of read pairs in the read pair group that support the alternate allele and are consistent with no isoforms in the set of isoforms.
Embodiment 72: The computer-implemented method of any one of embodiments 56-71, wherein the output includes a first count of read pairs in the read pair group that are consistent with the isoform and a second count of read pairs in the read pair group that are not consistent with the isoform.
Embodiment 73: The computer-implemented method of any one of embodiments 56-72, wherein the output includes a count of read pairs in the read pair group consistent with the isoform and supporting the reference allele.
Embodiment 74: The computer-implemented method of any one of embodiments 56-73, wherein the output includes a count of read pairs in the read pair group consistent with the isoform and supporting the alternate allele.
Embodiment 75: The computer-implemented method of any one of embodiments 56-74, wherein the output includes a count of read pairs in the read pair group that are exclusively consistent with the isoform and support the reference allele.
Embodiment 76: The computer-implemented method of any one of embodiments 56-75, wherein the output includes a count of read pairs in the read pair group that are exclusively consistent with the isoform and support the alternate allele.
Embodiment 77: The computer-implemented method of embodiment any one of embodiments 56-76, wherein the read pair group is derived from a diseased sample from a subject.
Embodiment 78: The computer-implemented method of any one of embodiments 56-77, wherein the read pair group is derived from cancer cells from a subject.
Embodiment 79: The computer-implemented method of any one of embodiments 56-78, wherein the output indicates RNA expression for a set of peptides and further comprising: designing, based on the output, a treatment that includes at least one of a peptide from the set of peptides, a precursor of the peptide, a nucleic acid that encodes the peptide, or a plurality of cells that express the peptide; and manufacturing the treatment.
Embodiment 80: The computer-implemented method of embodiment 79, wherein the peptide is selected from the set of peptides responsive to a determination that the peptide has at least a threshold level of RNA expression based on the output.
Embodiment 81: The computer-implemented method of embodiment 79 or embodiment 80, wherein the read pair group is derived from a disease sample of a subject such that the treatment is personalized for the subject.
Embodiment 82: The computer-implemented method of any one of embodiments 79-81, wherein the treatment is neoantigen treatment.
Embodiment 83: The computer-implemented method of any one of embodiments 79-82, wherein the treatment is a vaccine.
Embodiment 84: The computer-implemented method of any one of embodiments 56-83, further comprising: sequencing a disease sample from a subject to for the read pair group; identifying, based on at least one of the mutation-centric output or the isoform-specific output, one or more peptides having at least a threshold level of RNA expression; synthesizing mRNA that encode at least one peptide of the set of peptides; complexing the mRNA with lipids to developing an mRNA-lipoplex; and administering the mRNA-lipoplex to the subject.
Embodiment 85: A method for manufacturing a therapy, comprising: producing a vaccine comprising: one or more peptides; a plurality of nucleic acids that encode the one or more peptides; or a plurality of cells expressing the one or more peptides, wherein the one or more peptides are selected based on at least one of a mutation-centric output or an isoform-specific output generated by the method of any of embodiments 1-84, and wherein the one or more peptides are an incomplete subset of the set of peptides.
Embodiment 86: The method of embodiment 85, wherein the one or more peptides are selected as a set of peptides at least a threshold level of RNA expression based on the at least one of the mutation-centric output or the isoform-specific output.
Embodiment 87: The method of embodiment 85 or embodiment 86, wherein the vaccine comprises DNA that includes the plurality of nucleic acids, or RNA that includes the plurality of nucleic acids, optionally wherein the RNA is mRNA that includes the plurality of nucleic acids.
Embodiment 88: The method of embodiment 85 or embodiment 86, wherein the vaccine comprises the one or more peptides.
Embodiment 89: The method of any one of embodiments 85-88, wherein the vaccine is a tumor vaccine.
Embodiment 90: The method of any one of embodiments 85-89, wherein, for each peptide of the one or more peptides, the vaccine is a tumor comprising one or more of: a nucleotide sequence encoding the peptide, an amino acid sequence corresponding to the peptide, RNA encoding the peptide, DNA encoding the peptide, a cell expressing the peptide, or a vector encoding the peptide, optionally wherein the vector is a plasmid encoding the peptide.
Embodiment 91: The method of any one of embodiments 85-90, wherein the vaccine includes an individualized neoantigen specific therapy.
Embodiment 92: The method of any one of embodiments 85-91, wherein the vaccine comprises a plurality of cells expressing the one or more peptides.
Embodiment 93: A method comprising: collecting one or more biological samples from a subject, wherein the one or more biological samples includes a disease sample, and wherein the one or more biological samples is used to perform one or more of methods 1-92.
Embodiment 94: A computer-implemented method comprising: receiving, at a user device, input corresponding to a request to design an individualized vaccine for a subject; transmitting a communication to a remote system, the communication including an identifier of the subject, wherein the remote system is configured to perform one or more of the methods in embodiments 1-92 and transmit an output based on a corresponding result, and receiving the output generated based on the results.
Embodiment 95: A pharmaceutical composition comprising a nucleic acid sequence that encodes one or more peptides having been selected from among a set of peptides based on at least one of the mutation-centric output or the isoform-specific output generated by the method of any of embodiments 1-24, 36-44, and 56-78, wherein the one or more peptides are an incomplete subset of the set of peptides.
Embodiment 96: An immunogenic peptide identified based on the output generated by the method of any of embodiments 1-24, 36-44, and 56-78.
Embodiment 97: A nucleic acid sequence identified based on the output generated by the method of any of embodiments 1-78.
Embodiment 98; The nucleic acid sequence of embodiment 97, wherein the nucleic acid sequence includes a DNA sequence.
Embodiment 99: The nucleic acid sequence of embodiment 97, wherein the nucleic acid sequence includes an RNA sequence.
Embodiment 100: The nucleic acid sequence of embodiment 97, wherein the nucleic acid sequence includes an mRNA sequence.
Embodiment 101: A method of treating a subject comprising administering at least one of one or more peptides, one or more pharmaceutical compositions, or one or more nucleic acid sequences identified based on the output generated by the method of any of embodiments 1-24, 36-44, and 56-78.
Embodiment 102: A system comprising: one or more data processors; and a non-transitory computer readable storage medium containing instructions which, when executed on the one or more processors, cause the one or more data processors to perform part or all of one or more methods disclose embodiments 1-78.
Embodiment 103: A computer-program product tangibly embodied in a non-transitory machine-readable storage medium, including instructions configured to cause one of more data processors to perform part or all of one or more methods disclosed in embodiments 1-78.
Embodiment 104: A method comprising one or more methods disclosed in embodiments 1-94.

1. A computer-implemented method for quantifying ribonucleic acid (RNA) mutation expression, the method comprising:
identifying, for each read pair in a read pair group, a set of contiguously aligned regions and a splice junction configuration, wherein each read pair is within a selected range of a location of interest;
classifying each read pair in the read pair group based on the set of contiguously aligned regions and the splice junction configuration that correspond to each read pair, a reference genome, and a selected mutation; and
generating a mutation-centric output for the read pair group.

2. The computer-implemented method of claim 1, wherein the splice junction configuration for a read pair in the read pair group identifies at least one of a presence or a corresponding position of a splice junction in the read pair.

3. The computer-implemented method of claim 1, wherein classifying each read pair in the read pair group comprises:
classifying the read pair as supporting:
a reference allele when an allele at the location of interest matches the reference genome at the location of interest; or
an alternate allele when the allele at the location of interest matches the selected mutation at the location of interest; or
a null allele when the allele at the location of interest does not match either the reference genome or the selected mutation at the location of interest.

4. The computer-implemented method of claim 1, further comprising:
receiving sequence information for a plurality of read pairs; and
identifying a portion of the plurality of read pairs that fall within the selected range of the location of interest based on the sequence information to form the read pair group.

5. The computer-implemented method of claim 1, wherein the selected mutation is an indel and wherein classifying each read pair in the read pair group comprises:
identifying an alignment gap between two contiguously aligned regions within the read pair at the location of interest, wherein the alignment gap comprises at least one nucleotide that does not align with the reference genome and that is flanked by the two contiguously aligned regions.

6. The computer-implemented method of claim 1, wherein the selected mutation is a single nucleotide variation (SNV) and wherein classifying each read pair in the read pair group comprises:
classifying an allele at the location of interest within the read pair based on a nucleotide at the location of interest,
wherein the allele is classified as:
a reference allele if the nucleotide matches the reference genome at the location of interest; or
an alternate allele if the nucleotide matches the selected mutation at the location of interest; or
a null allele if the nucleotide does not match either the reference genome or the selected mutation at the location of interest.

7. The computer-implemented method of claim 1, wherein the selected mutation is a single nucleotide variation (SNV) and wherein classifying each read pair in the read pair group comprises:
classifying the read pair as a skip when the location of interest does not fall within a contiguously aligned region within the read pair due to deletion.

8. The computer-implemented method of claim 1, further comprising:
associating a read pair in the read pair group with an isoform derived from a transcript that includes the location of interest.

9. The computer-implemented method of claim 1, further comprising:
associating a read pair in the read pair group with an isoform derived from a transcript that includes the location of interest based on the set of contiguously aligned regions within the read pair and the splice junction configuration for the read pair.

10. The computer-implemented method of claim 1, further comprising:
associating a read pair in the read pair group with an isoform derived from a transcript that includes the location of interest when the splice junction configuration of the read pair is consistent with a set of isoform splice junctions within the isoform and the set of contiguously aligned regions within the read pair is overlapped by a set of exons within the isoform.

11. The computer-implemented method of claim 1, further comprising:
associating a read pair in the read pair group with an isoform derived from a transcript that includes the location of interest when:
the splice junction configuration of the read pair is consistent with a set of isoform splice junctions within the isoform; or
the set of contiguously aligned regions within the read pair is fully overlapped by a set of exons within the isoform.

12. The computer-implemented method of claim 1, wherein the mutation-centric output comprises
a count of read pairs in the read pair group that support a reference allele; or
a count of read pairs in the read pair group that support an alternate allele; or
a count of read pairs in the read pair group that support a null allele.

13. The computer-implemented method of claim 1, wherein the mutation-centric output comprises a count of read pairs in the read pair group that support a reference allele or an alternate allele and are consistent with at least one isoform derived from a transcript that includes the location of interest.

14. The computer-implemented method of claim 1, wherein the mutation-centric output comprises a count of read pairs in the read pair group that support a reference allele or an alternate allele and are consistent with no isoforms derived from a transcript that includes the location of interest.

15. The computer-implemented method of claim 1, further comprising:
determining to include an antigen that is derived from the selected mutation as a target for an immunotherapy responsive to the mutation-centric output indicating at least a threshold level of RNA expression for the selected mutation.

16. The computer-implemented method of claim 1, further comprising:
determining to exclude an antigen that is derived from the selected mutation as a target for an immunotherapy responsive to the mutation-centric output indicating that RNA expression for the selected mutation is below a threshold level.

17. The computer-implemented method of claim 15, wherein the immunotherapy is a target antigen-specific immunotherapy, wherein the target antigen-specific immunotherapy is a T cell therapy or a personalized cancer vaccine.

18. The computer-implemented method of claim 1, wherein the mutation-centric output indicates RNA expression for the selected mutation, the method further comprising:
determining that the selected mutation has at least a threshold level of RNA expression; and
developing a treatment that includes at least one of: a peptide that is derived from the selected mutation, a precursor of the peptide, nucleic acids that encode the peptide, or a plurality of cells that express the peptide.

19. A system comprising one or more processors and a memory coupled to the processors comprising instructions executable by the processors, the processors being operable when executing the instruction to:
identify, for each read pair in a read pair group, a set of contiguously aligned regions and a splice junction configuration, wherein each read pair is within a selected range of a location of interest;
classify each read pair in the read pair group based on the set of contiguously aligned regions and the splice junction configuration that correspond to each read pair, a reference genome, and a selected mutation; and
generate a mutation-centric output for the read pair group.

20. One or more computer-readable non-transitory storage media embodying software comprising instructions operable when executed to:
identify, for each read pair in a read pair group, a set of contiguously aligned regions and a splice junction configuration, wherein each read pair is within a selected range of a location of interest;
classify each read pair in the read pair group based on the set of contiguously aligned regions and the splice junction configuration that correspond to each read pair, a reference genome, and a selected mutation; and
generate a mutation-centric output for the read pair group.
